(19) United States
(12) Patent Application Publication
(10) Pub. No.: US 2026/0359976 A1
(43) Pub. Date: Nov. 12, 2026

(54) SYSTEMS AND METHODS FOR DETECTING TERTIARY LYMPHOID STRUCTURES

(71) Applicant: OWKIN INC., New York, NY (US)

(72) Inventors: Charles Maussion, Paris (FR); Lucie Gillet, Paris (FR); Jean-Eudes Le Douget, Paris (FR); Antoine Italiano, Bordeaux (FR); Francois Le Loarer, Bordeaux (FR); Isabelle Soubeyran, Bordeaux (FR)

(21) Appl. No.: 19/132,905

(22) PCT Filed: Nov. 29, 2023
(86) PCT No.: PCT/US2023/081687

§ 371 Date: May 27, 2025

(30) Foreign Application Priority Data
Nov. 30, 2022 (EP) 22306762.0

(51) Int. Cl. G06T 7/00; G06T 7/11; G06V 10/26; G06V 10/764; G06V 10/77; G06V 10/82; G06V 20/69; G16H 30/40

(52) U.S. Cl. CPC G06T 7/0012; G06T 7/11; G06V 10/267; G06V 10/764; G06V 10/7715; G06V 10/82; G06V 20/695; G06V 20/698; G16H 30/40; G06T 2207/10056; G06T 2207/20021; G06T 2207/20081; G06T 2207/20084; G06T 2207/30096

(57) ABSTRACT

A computer implemented method, a machine learning model, and a machine readable medium for detecting Tertiary Lymphoid Structures (TLSs) and/or the likelihood of the presence or absence of a TLS in a histology image and/or a subject are provided.

### FIELD OF INVENTION

This invention relates generally to machine learning and computer vision and more particularly to image preprocessing and classification.

### BACKGROUND OF THE INVENTION

Histopathological image analysis (HIA) is a critical element of diagnosis in many areas of medicine including oncology. Tertiary Lymphoid Structures (TLSs) are ectopic lymphoid structures developed at sites of chronic inflammation including tumors. TLSs exist under different maturation states in tumors, culminating in germinal center formation. The presence in the tumor compartment of mature TLS, characterized by mature follicles containing germinal centers, has been associated with improved survival upon cancer immunotherapies in patients with solid tumors. Accordingly, TLS could be used as a predictive factor of the patients who are more likely to benefit from immune checkpoint inhibitors. However, the pathological assessment of the TLS status remains time-consuming and usually requires additional analysis including immunohistochemical staining. Methods and systems for accurately and efficiently detecting TLSs in histology images and subjects are therefore needed.

### SUMMARY OF THE DESCRIPTION

A method and apparatus of a device that classifies an image is described.

In one aspect, disclosed herein is a computer-implemented method for detecting a presence or absence of tertiary lymphoid structures (TLSs) in a subject, comprising:
receiving a digitalized histology image of a sample obtained from the subject;
tiling the histology image into a set of tiles;
extracting a plurality of feature vectors from each of said tiles, wherein each feature of said one or more feature vectors represents local descriptors of the tile; and
classifying the histology image for a TLS status using at least the plurality of feature vectors and a classification model that is trained with a training set of histology images having known TLS annotations, wherein the TLS status indicates the presence or absence of a TLS in the subject.

In some embodiments, the classifying step comprises:
applying a first neural network to said one or more of the plurality of feature vectors, wherein said first neural network assigns a tile score to each tile of said set of tiles based on said one or more of the plurality of feature vectors, wherein the tile score represents a likelihood that said tile comprises a TLS; and
applying a second neural network to said each tile, wherein said second neural network aggregates subsets of said tile score of said set of tiles and determines the TLS status in the histology image, wherein:
said first neural network is trained using a training set of histology images comprising known local annotations of a presence or absence of TLSs at tile level; and
said first neural network and second neural network are trained using a training set of histology images comprising known global annotations of a presence or absence of a TLS at histology image level.

In some embodiments, said first neural network comprises a 1D convolutional layer. In some embodiments, said first neural network uses MoCo features as an input and outputs a likelihood that said tile comprises a TLS. In some embodiments, said second neural network comprises a Multi-Layer Perception model.

In some embodiments, the computer-implemented method provided herein further comprises
detecting one or more locations in which a TLS is present in said histology image.

In some embodiments of the computer-implemented methods provided herein, each tile comprises a plurality of pixels. In some embodiments, the method further comprises:
receiving a digitalized histology image of a sample obtained from the subject;
tiling the histology image into a set of tiles;
extracting segmentation mask from each tile;
applying a third neural network to each tile, wherein the third neural network detects pixel scores using extracted segmentation mask; and
detecting pixel-based TLS segmentation within the tile or the image, wherein said third neural network is trained using a training set of tiles comprising known pixel-based TLS segmentation mask within a tile.

In some embodiments, said third neural network assigns a pixel score to each pixel of each tile of said set of tiles and determines said pixel-based segmentation of a TLS within a tile, wherein the pixel score represents a likelihood that said pixel comprises a TLS. In some embodiments, said third neural network is a U-NET semantic segmentation neural network.

In some embodiments, said extracting step is performed by ResNet50 neural network. In some embodiments, said extraction of a plurality of feature vectors comprises Momentum Contrast (MoCo) or Momentum Contrast v2 (MoCo v2) algorithm.

In some embodiments of the computer-implemented method provided herein, said sample is a cancer. In some embodiments, the cancer is selected from the group consisting of lung cancer, sarcoma, bladder cancer, colorectal cancer, ovarian cancer, pancreatic cancer, and melanoma, kidney cancer, head and neck cancer, liver cancer, breast cancer, gastro-intestinal stromal tumor cancer, cervix cancer, endometrial cancer, stomach cancer, thyroid cancer, cholangiocarcinoma, prostate cancer, anal cancer, vulvar cancer, skin cancer, parotid cancer, digestive cancer, penile cancer, esophageal cancer, and cancer of unknown primary.

In some embodiments of the computer-implemented methods provided herein, the training set of tiles and/or training set of histology images are digitalized images of histology sections of cancers of heterologous origins (e.g., lung cancer, sarcoma, bladder cancer, colorectal cancer, ovarian cancer, pancreatic cancer, melanoma).

In some embodiments, the histology image is a digitalized whole slide image (WSI). In some embodiments, the digitalized histology image is a digitalized image of a histology section stained with a dye. In some embodiments, the dye is Haemotoxylin and Eosin (H&E).

In some embodiments, the classifying step further comprises:
sorting said set of tiles by
   picking tiles comprising highest TLS tile scores, and
   picking tiles comprising lowest TLS tile scores.

In some embodiments, the computer-implemented method provided herein comprises:
repeating all the steps of:
   receiving a digitalized histology image of a sample obtained from the subject;
   tiling the histology image into a set of tiles;
   extracting a plurality of feature vectors from each of said tiles, wherein each feature of said one or more feature vectors represents local descriptors of the tile; and
   classifying the histology image for a TLS status using at least the plurality of feature vectors and a classification model that is trained with a imaging training set having known TLS annotations
for each histology image in a plurality of histology images, and
processing the TLS status of the plurality of histology images, thereby detecting the presence or absence of a TLS in a subject.

In some embodiments, the histology image lacks local annotations of histopathological features. In some embodiments, each of said set of tiles comprise about 224×224 pixels.

In one aspect, disclosed herein is a machine readable medium having executable instructions to cause one or more processing units to perform a method of detecting a presence or absence of tertiary lymphoid structure (TLSs) in a subject, comprising:
receiving a digitalized histology image of a sample obtained from the subject;
tiling the histology image into a set of tiles;
extracting a plurality of feature vectors from each of said tiles, wherein each feature of said one or more feature vectors represents local descriptors of the tile; and
classifying the histology image for a TLS status using at least the plurality of feature vectors and a classification model that is trained with a training set of histology images having known TLS annotations, wherein the TLS status indicates the presence or absence of a TLS in the subject.

In some embodiments, the classifying step comprises:
applying a first neural network to said one or more of the plurality of feature vectors, wherein said first neural network assigns a tile score to each tile of said set of tiles based on said one or more of the plurality of feature vectors, wherein the tile score represents a likelihood that said tile comprises a TLS; and
applying a second neural network to said each tile, wherein said second neural network aggregates subsets of said tile score of said set of tiles and determines the TLS status in the histology image, wherein:
said first neural network is trained using a training set of histology images comprising known local annotations of a presence or absence of TLSs at tile level; and
said first neural network and second neural network are trained using a training set of histology images comprising known global annotations of a presence or absence of a TLS at histology image level.

In some embodiments, said first neural network comprises a 1D convolutional layer. In some embodiments, said first neural network uses MoCo features as an input and outputs a likelihood that said tile comprises a TLS. In some embodiments, said second neural network comprises a Multi-Layer Perception model.

In some embodiments, the method performed by the machine-readable medium provided herein further comprises
detecting one or more locations in which a TLS is present in said histology image.

In some embodiments of the method performed by the machine-readable medium provided herein, each tile comprises a plurality of pixels. In some embodiments, the method further comprises:
receiving a digitalized histology image of a sample obtained from the subject;
tiling the histology image into a set of tiles;
extracting segmentation mask from each tile;
applying a third neural network to each tile, wherein the third neural network detects pixel scores using extracted segmentation mask; and
detecting pixel-based TLS segmentation within the tile or the image, wherein said third neural network is trained using a training set of tiles comprising known pixel-based TLS segmentation mask within a tile.

In some embodiments, said third neural network assigns a pixel score to each pixel of each tile of said set of tiles and determines said pixel-based segmentation of a TLS within a tile, wherein the pixel score represents a likelihood that said pixel comprises a TLS. In some embodiments, said third neural network is a U-NET semantic segmentation neural network.

In some embodiments, said extracting step is performed by ResNet50 neural network. In some embodiments, said extraction of a plurality of feature vectors comprises Momentum Contrast (MoCo) or Momentum Contrast v2 (MoCo v2) algorithm.

In some embodiments, said extraction of a plurality of feature vectors comprises Momentum Contrast (MoCo) or Momentum Contrast v2 (MoCo v2) algorithm.

In some embodiments of the method performed by the machine-readable medium provided herein, said sample is a cancer. In some embodiments, the cancer is selected from the group consisting of lung cancer, sarcoma, bladder cancer, colorectal cancer, ovarian cancer, pancreatic cancer, melanoma, kidney cancer, head and neck cancer, liver cancer, breast cancer, gastro-intestinal stromal tumor cancer, cervix cancer, endometrial cancer, stomach cancer, thyroid cancer, cholangiocarcinoma, prostate cancer, anal cancer, vulvar cancer, skin cancer, parotid cancer, digestive cancer, penile cancer, esophageal cancer, and cancer of unknown primary.

In some embodiments of the method performed by the machine-readable medium provided herein, the training set of tiles and/or training set of histology images are digitalized images of histology sections of cancers of heterologous origins (e.g., lung cancer, sarcoma, bladder cancer, colorectal cancer, ovarian cancer, pancreatic cancer, melanoma).

In some embodiments, the histology image is a digitalized whole slide image (WSI). In some embodiments, the digitalized histology image is a digitalized image of a histology section stained with a dye. In some embodiments, the dye is Haemotoxylin and Eosin (H&E).

In some embodiments, the classifying step further comprises:
sorting said set of tiles by
   picking tiles comprising highest TLS tile scores, and
   picking tiles comprising lowest TLS tile scores.

In some embodiments, the method performed by the machine-readable medium provided herein comprises:
repeating all the steps of:
   receiving a digitalized histology image of a sample obtained from the subject;
   tiling the histology image into a set of tiles;
   extracting a plurality of feature vectors from each of said tiles, wherein each feature of said one or more feature vectors represents local descriptors of the tile; and
   classifying the histology image for a TLS status using at least the plurality of feature vectors and a classification model that is trained with a imaging training set having known TLS annotations
for each histology image in a plurality of histology images, and
processing the TLS status of the plurality of histology images, thereby detecting the presence or absence of a TLS in a subject.

In some embodiments, the histology image lacks local annotations of histopathological features. In some embodiments, each of said set of tiles comprise about 224×224 pixels.

### BRIEF DESCRIPTION OF THE DRAWINGS

The present invention is illustrated by way of example and not limitation in the Figures of the accompanying drawings in which like references indicate similar elements.

FIG. 1 illustrates an example flow diagram for a process of detecting the TLS status of an image and/or the presence or absence of a TLS in a subject using machine learning model, according to embodiments of the present disclosure.

FIG. 2 illustrates an example flow diagram for a process of classifying an image for the TLS status and optionally predicting a TLS location within the image and/or optionally predicting pixel-based TLS segmentation according to embodiments of the present disclosure.

FIG. 3 illustrates an example flow diagram for a process of training and validating a machine learning model for detecting the TLS status of an image and/or the presence or absence of a TLS in a subject according to embodiments of the present disclosure.

FIG. 4 depicts a receiver operating characteristic (ROC) curve for predicting the presence or absence of a TLS in a subject in cross-validation of the machine learning model according to the embodiments of the present disclosure.

FIG. 5 depicts an ROC curve for predicting the presence or absence of a TLS in a validation cohort of subjects using the machine learning model according to the embodiments of the present disclosure.

FIGS. 6A and 6B depict locations of TLSs in a histology image according to the embodiments of the present disclosure. FIG. 6A depicts an H&E stained histology image, with manual annotations by pathologists of TLS locations as marked by blue lines. FIG. 6B depicts TLS locations within a histology image as analyzed by the machine learning model, with the brighter areas having been assigned higher tile scores indicative of higher likelihood of a TLS.

FIG. 7 illustrates an example flow diagram for a process 700 that detects the pixel-based TLS segmentation in an image using machine learning model, according to embodiments of the present disclosure.

FIG. 8 depicts an example flow diagram for a process of training and validating a machine learning model for detecting the pixel-based TLS segmentation in an image, according to embodiments of the present disclosure.

FIGS. 9A and 9B depict pixel-wise TLS segmentation in a histology image. FIG. 9A depicts extractions of tiles and their segmentation masks from an H&E stained histology image having manual annotations by pathologists of TLS locations as marked by blue lines. FIG. 9B depicts a process of training a machine learning model to predict the TLS segmentation within a tile according to the embodiments of the present disclosure.

FIG. 10 illustrates an example of a computer system, which may be used in conjuncture with the embodiments described herein.

### DETAILED DESCRIPTION

Computer-implemented methods, associated systems, apparatus, and computer-readable media for detecting the presence or absence of tertiary lymphoid structures (TLSs) is described. In some aspects, provided herein is a diagnostic tool that applies machine learning to digital images of histology sections, e.g., whole slide images (WSI) to identify subjects who have or do not have TLSs associated with their tumors, and/or to aid in therapeutic decisions.

In the following description, numerous specific details are set forth to provide thorough explanation of embodiments of the present invention. It will be apparent, however, to one skilled in the art, that embodiments of the present invention may be practiced without these specific details. In other instances, well-known components, structures, and techniques have not been shown in detail in order not to obscure the understanding of this description.

Histology is the field of study relating to the microscopic features of biological specimens. Histopathology refers to the microscopic examination of specimens, e.g., tissues, obtained or otherwise derived from a subject, e.g., a patient, in order to assess a disease state. Histopathology specimens generally result from processing the specimen, e.g., tissue, in a manner that affixes the specimen, or a portion thereof, to a microscope slide. For example, thin sections of a tissue specimen may be obtained using a microtome or other suitable device, and the thin sections can be affixed to a slide. To assist in the visualization of the specimen, the specimen may optionally be further processed, for example, by applying a stain. Many stains for visualizing cells and tissues have been developed. These include, without limitation, Haemotoxylin and Eosin (H&E), methylene blue, Masson's trichome, Congo red, Oil Red O, and safranin. H&E is routinely used by pathologists to aid in visualizing cells within a tissue specimen. Hematoxylin stains the nuclei of cells blue, and eosin stains the cytoplasm and extracellular matrix pink. A pathologist visually inspecting an H&E stained slide can use this information to assess the morphological features of the tissue. However, H&E stained slides generally contain insufficient information to assess the presence or absence of particular biomarkers by visual inspection. Visualization of specific biomarkers (e.g., protein or RNA biomarkers) can be achieved with additional staining techniques which depend on the use of labeled detection reagents that specifically bind to a marker of interest, e.g., immunofluorescence, immunohistochemistry, in situ hybridization, etc. Such techniques are useful for determining the expression of individual genes or proteins, but are not practical for assessing complex expression patters involving a large number of biomarkers. Global expression profiling can be achieved by way of genomic and proteomic methods using separate samples derived from the same tissue source as the specimen used for histopathological analysis. Notwithstanding, such methods are costly and time consuming, requiring the use of specialized equipment and reagents, and do not provide any information correlating biomarker expression to particular regions within the tissue specimen, e.g., particular regions within the H&E stained image.

“Tertiary lymphoid structures” (TLSs) as used herein refer to ectopic lymphoid structures that develop in non-lymphoid tissues at sites of in chronic inflammation, including autoimmune diseases, transplant rejection, and cancer. TLS are similar to lymph nodes in both structure and development, and the organization and integrity of TLS are supported by stromal cells. TLSs comprise a heterogeneous population of cells, including B cells (e.g., germinal center B cells), T cells (e.g., type 1 T helper (Th1) cells, T follicular helper (Tfh) cells, regulatory T (Treg) cells), dendritic cells (e.g., follicular dendritic cells (FDCs), mature dendritic cells), plasma cells, neutrophils, and macrophages. TLSs are also characterized by the presence of high endothelial venules (HEVs), which refer to blood vessels adapted for lymphocyte trafficking. Well-developed TLS contain B-cell follicles with actively replicating B-cell germinal centers surrounded by a T-cell region. A “germinal center” as used herein refers to a transiently formed microstructure within B cell zone (follicles) in secondary lymphoid organs where mature B cells are activated, proliferate, differentiate, and mutate their antibody genes. Interspersed throughout the TLS are high endothelial venules and dendritic cell-lysosomal associated membrane protein (DC-LAMP) and dendritic cells. TLS are not encapsulated and occur within various nonlymphoid tissues, such as epithelial tissues and stroma. In contrast to secondary lymphoid organs (SLOs), which are well defined and refer to specific structures such as lymph nodes, TLS refer to structures with varying organization. TLS can be simple lymphocyte aggregates or more organized structures present in a nonlymphoid structure (Munoz-Erazo, L. et al. 2020 Cell. Mol. Immnol. 17:570-575).

The use of TLSs as a prognostic indicator of cancer has been proposed (Colbeck, E. J. et al. 2017 Front. Immunol. 8, 1830; Trajkovski, G. et al. 2018 Open Access Maced. J. Med. Sci. 6, 1824-1828). TLS presence or induction following cancer therapies can predict therapeutic responses, usually correlating with favorable therapeutic responses and/or clinical outcome. TLS presence or induction can also be a prognostic factor, usually indicating good prognosis in various cancers In some embodiments, such correlation between TLS presence or induction and favorable therapeutic response, clinical outcome, or prognosis is absent in hepatocellular carcinoma (HCC). For example, The presence in the tumor compartment of mature TLS, characterized by mature follicles containing germinal centers, has been associated with improved survival upon cancer immunotherapies for patients with solid tumors. Therefore, TLS can be used to identify subjects who are more likely to benefit from immune checkpoint inhibitors. The number, density, and location of TLSs can affect the subject's prognosis or response to treatment (Munoz-Erazo, L. et al. 2020 Cell. Mol. Immnol. 17:570-575).

Further, exogenous induction of TLS may provide a therapeutic benefit. TLS formation through various pharmacological approaches may promote lymphocyte infiltration, activation by tumor antigens, and differentiation to increase the antitumor immune response, and/or increase the sensitivity of immune cold tumors to immunotherapies when used in conjunction with immune checkpoint blockade, vaccines, viruses, local intratumoral agents, or intervention therapies. In immunologically active tumors with a disturbed tumor microenvironment and strong chronic inflammation, angiogenesis, and a fibrotic stroma, the use of antiangiogenic and anti-immunosuppressive agents may help normalize the immune contexture, favoring TLS formation and therapeutic response to immune checkpoint blockade. Several approaches are being developed using chemokines, cytokines, antibodies, antigen-presenting cells or synthetic scaffolds to induce TLS formation. Strategies aiming to induce TLS neogenesis in immune-low tumors and in immune-high tumors, in this case, in combination with therapeutic agents dampening the inflammatory environment and/or with immune checkpoint inhibitors, represent promising avenues for cancer treatment.

Detection of TLSs, including their locations and amount, has been performed based on morphological assessment of TLSs, relying on local annotation of regions of significance within the image by an expert pathologist. For example, H&E staining enables the detection of TLSs in formalin-fixed paraffin-embedded tumor sections. Mature TLSs correspond to lymphoid follicles including a dense cellular aggregate resembling germinal centers found in secondary lymphoid structures (SLOs). Less differentiated structures such as lymphoid aggregates and lymphoid follicles without germinal centers can also be detected by pathological examination of H&E-stained histology slides. Currently, such pathological detection and assessment of TLSs remains time consuming and labor intensive.

Additional analysis including immunohistochemistry can be used to aid TLS detection. Immunohistochemistry (IHC) on consecutive tumor sections or double or multiplex labelling techniques using the markers present in cells or tissues comprising TLS can also be used to detect TLSs, followed by evaluation of TLS density, size and, cellular content on scanned images, e.g., using quantitative digital pathology software. Common cell types present in TLSs and exemplary markers that can be used to detect TLSs are set forth in Table 1 below.

TABLE 1
Cell Types Present in TLS and Their Markers

| Cell Types | Markers |
|---|---|
| B cells | CD20, CD19 |
| Cell Types | Markers |
| Germinal center | Activation-induced deaminase (AID), Ki67, |
| B cells | CD21 (CR2), CD35 (CR1), CD23 (FcerII), |
| | CD269, CD138 (weak expression on late- |
| | stage germinal B cells) |
| Follicular dendritic | CD21 (CR2), CD35 (CR1) and CD23 (FcerII) |
| cells (FDCs) | |
| Plasma cells | CD138 |
| T cells | CD3, CD8, CD4 |
| Type 1 T helper | PD1 |
| (Th1) cells | |
| T follicular helper | FOXP3 |
| (Tfh) cells, regulatory | |
| T (Treg) cells | |
| Dendritic cells (DCs) | DY lysosome-associated membrane |
| | glycoprotein (DC-LAMP) |
| Mature dendritic cells | CD83, CD86 |
| Neutrophils | CD66b, myeloperoxidase |
| Macrophages | CD68 |
| High endothelial venules | Peripheral node addressin (PNAd), MECA79 |

Various gene signatures of TLSs can also be used to aid pathological detection of TLSs in a sample, e.g., a histology sample of a cancer. Exemplary gene signatures of TLS are provided in Table 2 below. The characteristics, functions, roles, and implications of TLSs have been discussed in the following literature, which is herein incorporated by reference in its entirety: Sautès-Fridman et al. 2019 Nat. Rev. Cancer 19(6); Sautès-Fridman et al. 2016 Front. Immunol. 7:407; Vanherseche et al. 2021 Nat. Cancer 2(8):794-802.

TABLE 2
Gene signatures for the detection of TLS

| | | |
|---|---|---|
| | | TLS-related functions and cancer types in which the |
| Gene name | Protein aliases | signature has been used |

12-Chemokine signature in colorectal cancer, melanoma, hepatocellular carcinoma and breast cancer

| | | |
|---|---|---|
| CCL2 | MCP1 or MCAF | Monocyte, immature DC and T cell chemotaxis; cell |
| | | adhesion; and cellular response to IFNγ, IL-1 and IL-6 |
| CCL3 | MIP1α | Monocyte and T cell chemotaxis and cellular response to |
| | | IFNγ, TNF and IL-1 |
| CCLA | MIP1β or LAG1 | Monocyte, neutrophil and T cell chemotaxis; cell adhesion; |
| | | cellular response to IFNγ, TNF and IL-1; and inflammatory |
| | | response |
| CCL5 | RANTES | Monocyte, neutrophil and T cell chemotaxis; cell adhesion; |
| | | cellular response to IFNγ, TNF and IL-1; and inflammatory |
| | | response |
| CCL8 | MCP2 or HC14 | Monocyte, neutrophil and T cell chemotaxis; cell adhesion; |
| | | cellular response to IFNγ, TNF and IL-1; and chronic |
| | | inflammatory response |
| CCL18 | PARC, MIP4, | Monocyte, neutrophil and T cell chemotaxis; cell adhesion; |
| | AMAC1 or DCCK1 | cellular response to IFNγ, TNF and IL-1; and inflammatory |
| | | response |
| CCL19 | MIP3β or ELC | Mature DC and T cell chemotaxis; T cell co-stimulation; cell |
| | | maturation; cellular response to IFNγ, TNF and IL-1; |
| | | inflammatory response; establishment of T cell polarity; |
| | | immunological synapse formation; and positive regulation of |
| | | IL-1β, IL-12 and TNF secretion |
| CCL21 | SLC, 6Ckine or TCA4 | Mature DC and T cell chemotaxis; T cell co-stimulation; cell |
| | | maturation; cellular response to IFNγ, TNF and IL-1; |
| | | inflammatory response; establishment of T cell polarity; |
| | | negative regulation of DC dendrite assembly; positive |
| | | regulation of DC antigen presentation function; and cellular |
| | | response to IL-1 and TNF |
| CXCL9 | MIG or CMK | Neutrophil and T cell chemotaxis; T_{H}l cell polarization; |
| | | inflammatory response; and regulation of cell proliferation |
| CXCL10 | IP10 | Neutrophil, monocyte and T cell chemotaxis; T_{H}1 cell |
| | | polarization; inflammatory response; and positive regulation |
| | | of cell proliferation |
| CXCL11 | IP9 or ITAC | T cell chemotaxis; T_{H}1 cell polarization; inflammatory |
| | | response; and positive regulation of cell proliferation |
| CXCL13 | BLC, BCA1 or | B cell and T_{FH} cell chemotaxis; germinal center formation; |
| | CCYB13 | lymph node development; regulation of humoral immunity; |
| | | and regulation of cell proliferation |

Tfh cell signature in breast cancer

| | | |
|---|---|---|
| CXCL13 | BLC, BCA1 or | B cell and T_{FH} cell chemotaxis; germinal center formation; |
| | SCYB13 | lymph node development; regulation of humoral immunity; |
| | | and regulation of cell proliferation |
| CD200 | OX2 | Regulation of immune response; negative regulation of |
| | | macrophage activation; and cell recognition |
| FBLN7 | FLJ37440 or TM14 | Cell adhesion |
| ICOS | CD278 | T cell co-stimulation and T cell tolerance induction |
| SGPP2 | SPP2 or SPPase2 | Regulation of immune response; positive regulation of signal |
| | | transduction; and positive regulation of natural killer cell- |
| | | mediated cytotoxicity |
| SH2D1A | NA | Signal transduction of T and B cell signal activation |
| TIGIT | VSTM3 or VSIG9 | T cell co-inhibitory receptor; negative regulation of IL-12 |
| | | production; and positive regulation of IL-10 production |
| PDCDI | PD1 | T cell co-stimulation and positive regulator of T cell |
| | | apoptotic process |

Th1 cell and B cell signature in gastric cancer

| | | |
|---|---|---|
| CD4 | NA | T cell activation, differentiation and selection; and cytokine |
| | | production |
| CCR5 | CD195 | Receptor for CCL3, CCL4 and CCL5; myeloid and |
| | | lymphocyte chemotaxis; inflammatory response; and positive |
| | | regulation of IL-1, IL-6 and TNF production |
| CSCR3 | CD182, CD183 or | Receptor for CXCL9, CXCL10 and CXCL11; neutrophil and |
| | GPR9 | T cell chemotaxis; T_{H}1 cell polarization; inflammatory |
| | | response; and cell adhesion |
| CSF2 | GM-CSF | DC differentiation; macrophage activation; negative |
| | | regulation of cytolysis; positive regulation of cell |
| | | proliferation; and positive regulation of IL-23 secretion |
| IGSF6 | DORA | Immune response |
| IL3RA | CD25, IL-2R or p55 | Positive regulation of activated T cell proliferation; positive |
| | | regulation of T cell differentiation; inflammatory response; |
| | | and regulation of cell tolerance induction |
| CD38 | ADPRC1 | T cell activation; positive regulation of B cell proliferation; B |
| | | cell receptor signaling; cellular defense response; cell |
| | | adhesion; and response to IL-1 |
| CD40 | TNFRSF5 | B cell proliferation; inflammatory response; positive |
| | | regulation of IL-12 production; isotype switching to IgG; and |
| | | immunoglobulin secretion |
| CD5 | LEU1 | T cell co-stimulation and cell proliferation |
| MS4A1 | CD20 or LEU16 | Positive regulation of B cell lineage; B cell proliferation; and |
| | | humoral immune response |
| SDC1 | SDC, CD138 or | Cell migration and inflammatory response |
| | syndecan | |
| GFI1 | ZNF163 or SCN2 | Regulation of transcription |
| IL1R1 | IL-1RA, IL-1R or | Cell surface receptor signaling; regulation of inflammatory |
| | CD121A | response; and response to TGFβ |
| IL1R2 | IL-1RB or CD121B | Inflammatory response |
| IL10 | TGIF, GVHDS or | Positive regulation of B cell differentiation; |
| | CSIF | inflammatory response; negative regulation of T and B |
| | | cell proliferation; negative regulation of IFNγ, IL-1, IL-6, |
| | | IL-8, IL-12, IL-18 and TNF production; negative |
| | | regulation of myeloid DC activation; and regulation of |
| | | isotype switching |
| CCL20 | MIP3α, LARC or | Immature DC, monocyte, neutrophil and T cell |
| | Exodus | chemotaxis; cellular response to IL-1, TNF and LPS; |
| | | and inflammatory response |
| IRF4 | MUM1 or LSIRF | T cell activation; TH17 cell polarization; positive |
| | | regulation of IL-2, IL-4, IL-10 and IL-13 production; |
| | | and regulation of TH cell differentiation |
| TRAF6 | RNF85 | T cell receptor signaling; TH1 cell immune response; |
| | | antigen processing and presentation; myeloid DC |
| | | differentiation; positive regulation of T cell proliferation, |
| | | activation and cytokine production; positive regulation of |
| | | IL-12 production; and response to IL-1 |
| STAT5A | MGF | Regulation of transcription |

Plasma cell signature in ovarian cancer

| | | |
|---|---|---|
| TNFRSF17 | BCM, BCMA, | Adaptive immune response; response to TNF; regulation of |
| | CD269 or | humoral immunity; and B cell survival |
| | TNFRSF13A | |

CXCL13 signature in colorectal cancer and soft tissue sarcoma

| | | |
|---|---|---|
| CXCL13 | BLC, BCA1 or | B cell and TFH cell chemotaxis; germinal center |
| | SCYB13 | formation; lymph node development; regulation of |
| | | humoral |

Even using histology analysis with IHC or gene signature analysis provided herein, detection of TLS remains slow, laborious, and expensive, and thus is not well suited to high-throughput applications. In order to overcome this problem, the present disclosure provides an image processing pipeline analyze a histopathology image without the use of local annotations. This pipeline is initially based on segmenting a large image (e.g. WSI) into smaller images, e.g., 224×224 pixel images, and detecting a region of interest within the image on which to perform classification with Otsu's method. Thus, this classification works on small images, which are far less computationally expensive than a single large image. These smaller images can be fed to a ResNet-type convolutional neural network to extract a feature vector from each small image, where the feature vector comprises local descriptors for that small image. A score is computed for each small image from the extracted feature vectors, as a local tile level (instance) descriptor. The top and bottom instances are used as input to a Multi-Layer Perceptron (MLP) to perform classification on them.

In one embodiment, the device classifies a histology image (e.g., cancer histology image) using one or more neural network models to determine a label for that image. In this embodiment, the histology image can be a large image, where it is computationally impractical to process the image as a whole solely using a neural network model. In particular, the device reduces the amount of computing resources (e.g., time and/or memory requirements) needed to perform the image classification task on these large images. Such a reduction of resources further improves the performance of the device when executing the image classification task. In addition, the device can classify a whole-slide image (WSI), even when this type of image is too large to fit in the memory of a graphics processing unit commonly used to train machine learning models. In a further embodiment, the device reduces the dimensionality of the data, thus giving better generalization error and is more efficient in terms of model accuracy.

Reference in the specification to “one embodiment” or “some embodiments” means that a particular feature, structure, or characteristic described in connection with the embodiment can be included in at least one embodiment of the invention. The appearances of the phrase “in one embodiment” in various places in the specification do not necessarily all refer to the same embodiment. The term “exemplary” is used herein in the sense of “example,” rather than “ideal.” From this disclosure, it should be understood that the invention is not limited to the examples described herein.

For any methods described herein, the ordering of steps as presented, whether in the text or in an accompanying flow diagram, should not be taken to mean that those steps must be performed in the order presented, unless otherwise specified or required by context. Rather, the order of steps presents one embodiment of the methods provided, and in general such steps may alternatively be performed in a different order or simultaneously. The processes depicted in the Figures that follow may be performed by processing logic that comprises hardware (e.g., circuitry, dedicated logic, etc.), software (such as is run on a general-purpose computer system or a dedicated machine), or a combination of both. Although the processes are described below in terms of some sequential operations, it should be appreciated that some of the operations described may be performed in different order. Moreover, some operations may be performed in parallel rather than sequentially.

Computing methods used for implementing the methods provided herein can include, for example, machine learning, artificial intelligence (AI), deep learning (DL), neural networks, classification and/or clustering algorithms, and regression algorithms.

The terms “server,” “client,” and “device” are intended to refer generally to data processing systems rather than specifically to a particular form factor for the server, client, and/or device.

Reference in the specification to “local annotation(s)” means metadata (e.g., text, marking, number, and/or another type of metadata) that applies to part of an image, and not to the image as a whole. For example, in one embodiment, a local annotation can be a marking of a region of interest in an image, such as a histology image. Exemplary local annotations include markings outlining or otherwise identifying a portion of the image, e.g., a tumor region of the image, a stromal region of the image, identification of cell types within the image, identification of biological structures composed of multiple cells in the image, e.g., TLSs. In contrast, reference in the specification to “global annotation(s)” means metadata applied to the image as a whole. Exemplary global annotations include a label identifying the image as a whole, data regarding how the image was acquired, a label identifying a feature of the subject from whom the image is derived, e.g., a label indicating the age, sex, diagnosis, etc. of the subject from whom the image is derived, and/or any other data applying to the image as a whole. In some embodiments, a global annotation can indicate the presence, amount, or location of TLSs known or understood to be present in the subject from whom the image is derived. In other embodiments, a global annotation can indicate a known characteristic of the subject from whom the image is derived, such as duration of survival (e.g., duration of survival following acquisition of the sample represented in the image) or response to a given treatment. In some embodiments described herein, images may be used that contain global annotations, in the absence of local annotations.

A “patient” refers to a subject who shows symptoms and/or complications of a disease or condition (e.g., malignant tumor, cancer), is under the treatment of a clinician (e.g., an oncologist), has been diagnosed as having a disease or condition, and/or is at a risk of developing a disease or condition. The term “patient” includes human and veterinary subjects. Any reference to subjects in the present disclosure should be understood to include the possibility that the subject is a “patient” unless clearly dictated otherwise by context.

As used herein, a “subject” is an animal, such as a mammal, including a primate (such as a human, a monkey, and a chimpanzee) or a non-primate (such as a cow, a pig, and a horse) that benefits from the methods according to the present disclosure. In some aspects of the invention, the subject is a human, such as a human diagnosed with cancer. The subject may be a female human. The subject may be a male human. In some aspects, the subject is an adult subject.

As used herein, “predict” or “predicting” in the context of this disclosure refers to determining a likelihood of presence or absence of a condition (e.g., TLSs) in the past, present, or future. In some embodiments, a model (e.g., 1D convolutional layer with MoCo features followed by Multi-Layer Perception) can predict a likelihood of TLS status by one or more of the following measures of test accuracy:
an odds ratio greater than 1, preferably about 2 or more or about 0.5 or less, about 3 or more or about 0.33 or less, about 4 or more or about 0.25 or less, about 5 or more or about 0.2 or less, or about 10 or more or about 0.1 or less;
a specificity of greater than 0.5, preferably at least about 0.6, at least about 0.7, at least about 0.8, at least about 0.9, or at least about 0.95, with a corresponding sensitivity greater than 0.2, preferably at least about 0.3, at least about 0.4, at least about 0.5, at least about 0.6, at least about 0.7, at least about 0.8, at least about 0.9, or at least about 0.95;
a sensitivity of at least 0.5, preferably at least about 0.6, at least about 0.7, at least about 0.8, at least about 0.9, or at least about 0.95, with a corresponding sensitivity at least 0.2, preferably at least about 0.3, at least about 0.4, at least about 0.5, at least about 0.6, at least about 0.7, at least about 0.8, at least about 0.9, or at least about 0.95;
at least about 75% sensitivity, combined with at least about 75% specificity;
a positive likelihood ratio [calculated as sensitivity/(1−specificity)] of greater than 1, preferably at least about 2, at least about 3, at least about 4, at least about 5, at least about 10; or
a negative likelihood ratio [calculated as (1−sensitivity)/specificity] of less than 1, preferably about 0.5 or less, about 0.33 or less, about 0.25 or less, or about 0.1 or less.

As used herein, a “tumor” refers to an abnormal growth of cells or a tissue and/or a mass resulting therefrom. In some embodiments, a tumor tissue or tumor cells are malignant (e.g., cancerous). “Cancer” or “malignant tumor” as used herein refers to a plurality of cells in which abnormal cells divide without control and with an ability to invade or metastasize to neighboring or distant tissues or organs. Exemplary malignant tumors or cancers include lung cancer, sarcoma, bladder cancer, colorectal cancer, ovarian cancer, pancreatic cancer, and melanoma.

### I. Method of Detecting TLSs in a Subject

In some aspects, provided herein is a computer-implemented method for detecting the presence or absence or TLSs in a subject. In one aspect, the computer-implemented method comprises:
receiving a digitalized histology image of a sample obtained from the subject;
tiling the histology image into a set of tiles;
extracting a plurality of feature vectors from each of said tiles, wherein each feature of said one or more feature vectors represents local descriptors of the tile; and
classifying the histology image for a TLS status using at least the plurality of feature vectors and a classification model that is trained with a imaging training set having known TLS annotations, wherein the TLS status indicates the presence or absence of a TLS in the subject. In some embodiments, the histology image lacks local annotations of histopathological features and/or global annotations of TLS status.

As used herein, a “tile” refers to a subsection of an image. “Tiling” as used herein refers to dividing an image or a region of interest into tiles.

A used herein, the term “digitalized image” or “digital image” refers to an electronic image represented by a collection of pixels which can be viewed, processed and/or analyzed by a computer. In some aspects of the present disclosure, digital images of histology slides, e.g., H&E stained slides, allow computational assessment of tissue specimens, in addition to or alternatively to visual inspection by a pathologist. In some embodiments, a digital image can be acquired by means of a digital camera or other optical device capable of capturing digital images from a slide, or portion thereof. In other embodiments, a digital image can be acquired by means of scanning a non-electronic image of a slide, or portion thereof. In some embodiments, the digital image used in the applications provided herein is a whole slide image. As used herein, the term “whole slide image (WSI),” refers to an image that includes all or nearly all portions of a tissue section, e.g., a tissue section present on a histology slide. In some embodiments, a WSI includes an image of an entire slide. In other embodiments, the digital image used in the applications provided herein is a selected portion of a tissue section, e.g., a tissue section present on a histology slide. In some embodiments, a digital image is acquired after a tissue section has been treated with a stain, e.g., H&E.

A “TLS” status as used herein refer to the presence, location, or amount of TLSs in a subject, a histology image, a tile within the histology image, or a pixel within the tile. TLS status can be expressed in binary form (e.g., present or absent), as classification, as continuous range (e.g., amount expressed by numbers), as description (e.g., narrative description of location or abundance of TLSs), or a combination of any thereof. TLS status can be based on the probability score assigned to the subject, histology image, tile, or pixel, or components thereof. For example, at the subject level, a TLS status can refer to the presence or absence of TLSs in the subject, e.g., at the site of cancer, and can be determined based on the probability score (e.g., image score) assigned to the histology images of samples (e.g., tumor samples) obtained from the subject. At the histology image level, a TLS status can refer to the presence or absence, the amount, or the location (e.g., segmentation within the image) of TLSs in the image, and can be determined based on the image score assigned to the image or tile score assigned to the tiles within the image. At the tile level, a TLS status can refer to the presence or absence, the amount, or the location (e.g., segmentation within the tile) of TLSs in the tile, and can be determined based on the tile score assigned to the tile or the pixel score assigned to the pixels within the tile. At the pixel level, a TLS status can refer to the presence or absence or the amount of TLSs in the pixel, and can be determined based on the pixel score assigned to the pixel.

As used herein, “a “score”, “probability score”, or “risk score” refers to a likelihood of a certain condition, e.g., a TLS is present, or present in therapeutic relevance (e.g., present at least in a certain amount that is of clinical relevance, present at least in a certain location that is of clinical relevance). In some embodiments, the score is expressed as a classification. In other embodiments, the score is expressed as a continuous range. In one embodiment, the score represents the likelihood that TLSs are present in the pixel, the tile, the image, or the subject.

According to one embodiment, the device classifies at least one histology input image by segmenting the image between at least one region of interest containing information useful for classification and at least one background region containing little or no information useful for classification, by applying a first convolutional neural network. The device further tiles this region of interest of the image into a set of tiles. In addition, the device extracts a feature vector for each tile by applying a second convolutional neural network, where the features are local descriptors of the tile. Furthermore, the device processes the extracted feature vectors of the tiles in order to classify the image. In one embodiment, by segmenting the input image, the device processes a reduced number of tiles and avoids a processing of the whole image.

In one embodiment, the first convolutional network is a semantic segmentation neural network classifying the pixels of the input image as one of the following two categories: (a) Region of interest; and (b) Background region. Further, the tiling step (b) can be performed by applying a fixed tiling grid to the image, so that said tiles have a predetermined size. In addition, at least one level of zoom can be applied to the tiles. For example, multiple levels of zoom can be applied to the tiles and tiles at different levels of zoom are combined. In addition, the device can optionally randomly sample the tiles and/or pad the set of tiles with blank tiles, so that the set of tiles comprises a given number of tiles.

In a further embodiment, the second convolutional neural network can be a residual neural network, such as a ResNet50 residual neural network or a ResNet101 residual neural network with the last layer removed using the previous layer as output, or a VGG neural network. This second convolutional neural network can be a pre-trained neural network, allowing the use of a state-of-the-art advanced neural network, without needing to have a large-scale image database and the computational resources to train this neural network.

In one embodiment, the device can compute at least one score of the tile from the extracted feature vector, where the tile score is representative of a contribution of the tile into the classification of the image. With the tile scores, the device can sort the set of the tile scores and select a subset of the tile scores based on their value and/or their rank in the sorted set; and applying a classifier to the kept tile scores in order to classify the image. The device can further apply this classification to multiple input images, where the device can aggregate groups of corresponding tiles from the different input images.

In an alternative embodiment, the device can also aggregate clusters of neighboring tiles. In this embodiment, aggregating a cluster of tiles can include concatenating the tiles of the cluster, selecting a single tile from the cluster according to a given criterion, using the cluster as a multidimensional object, or aggregating the values for example through a mean or a max pooling operation. In addition, the device can apply an autoencoder on the extracted feature vectors so as to reduce the dimensionality of the features. In one embodiment, the image can be a histopathology slide, the region of interest being a tissue region, and the classification of the image being a diagnosis classification.

In an alternative embodiment, when local annotations are available, such as the presence of tumors in regions of the slides, a hybrid technique can be used to take those annotations into account. To do so, the device can train the machine learning model for two concurrent tasks: (1) the local prediction of the presence of tumors and/or other macroscopic properties on each tile and the prediction of a set of global labels. A complex architecture can be used by the device (or multiple devices) that involves, on one side, the classification system described above to process a set of 2,048 features. On the other side, the device applies a convolutional neural network to transform the features of the N tiles into an N*2,048 features vector. Based on this vector, the device trains a convolutional neural network to predict, for each tile, the presence or absence of tumor (or some other macroscopic property). The device can take both the output of the prediction and the N*2,048 features vector and apply an operation of weighted pooling on the concatenation of those two vectors to get a 2,048 features vector for the input image. The device concatenates the classification model's output and the 2,048 features obtained and try to predict based on this vector, a set of global labels for that image (e.g., survival, tumor size, necrosis, and/or other types of predictions). The loss of the model involves both global and local predictions. In this embodiment, by adding information derived from the local annotations into the computational flow, the performance of the overall model can be increased.

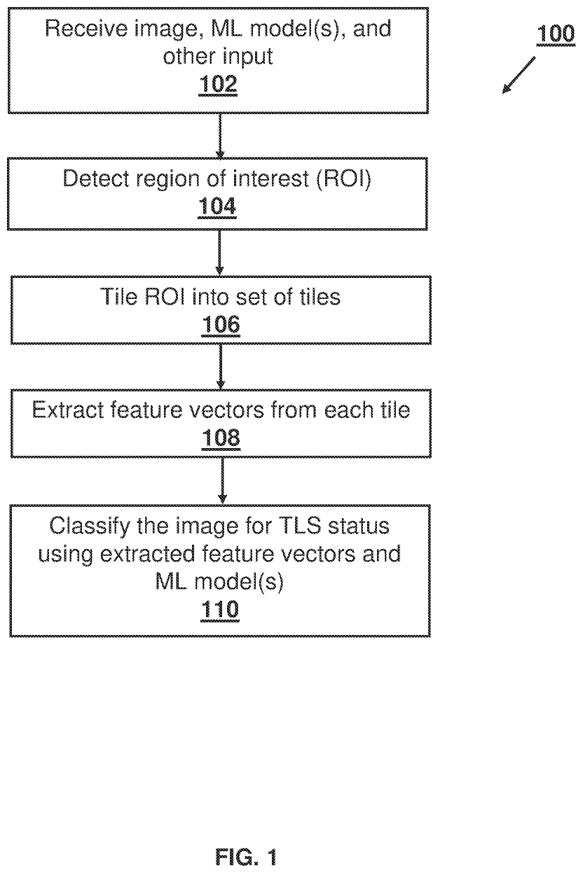

FIG. 1 illustrates an example flow diagram for a process 100 that detects the TLS status of an image and/or the presence or absence of a TLS in a subject using machine learning model, according to embodiments of the present disclosure. At block 102, process 100 receives an image, one or more machine learning (ML) models, and optionally other input. In some embodiments, the image is a digitalized histology image of a sample obtained from the subject, such as a digitalized whole slide image (WSI).

At block 104, process 100 detects region of interest (ROT) within the image. As used herein, the “region of interest” (ROI) of an image could be any region semantically relevant for the task to be performed, in particular regions corresponding to tissues, organs, bones, cells, body fluids, etc. when in the context of histopathology. In a further embodiment, process 100 segments the image into a region of interest and a background region. In this embodiment, by extracting a region of interest from the input image can decrease the amount of computation needed to classify the input image. For example and in one embodiment, histopathology slides (or other types of images) can include empty region(s) of the image with little or no tissue at all, thus it is useful to introduce what is called a “tissue detection” or “matter detection” method in order to evaluate if a region of the slide contains any tissue. More generally, when the goal is to classify a large image, it is relevant to identify regions of interest in the image and differentiate them from background regions. These regions of interest are the regions of an image containing valuable information for the classification process. In addition, the background regions are areas of the image that include little or no valuable information, where the background regions could be considered as noise for the task at hand. In order to realize this task, various different types of image segmentation schemes can be used. For example and in one embodiment, Otsu's method can be used to segment the image, where Otsu's method is a simple thresholding method based on the intensity histogram of the image. In this embodiment, segmenting the image using Otsu's method has shown pretty good results when the image contains two classes of pixels following a bimodal distribution, for example foreground pixels and background pixels or, more specifically tissue and non-tissue. However, this method is known to perform badly on complex images when the histogram of intensity level cannot be assumed to have a bimodal distribution. This calls for a more robust technique in order to improve the overall efficiency of the method.

In another embodiment, and in order to improve the robustness of the image segmentation and to be able to tackle complex images (such as histopathology images), a semantic segmentation neural network can be used to segment the images, such as a U-NET semantic segmentation neural network, a SegNet, a DeepLab or another type of semantic segmentation neural network. In this embodiment, a semantic segmentation neural network can be used that does not depend on a particular distribution in the intensity histogram. Moreover, using such a neural network allows the image segmentation to take into account multichannel images such as Red-Green-Blue (RGB) images. Thus, the segmentation does not just rely on the histogram of pixel intensity but can take advantage of the semantics of the image. In one embodiment, the semantic segmentation neural network is trained to segment the tissue of the histopathology image from the background of this image, so as to differentiate a stained or unstained tissue from a background.

In another embodiment, another advantage of using a U-NET segmentation neural network is that this network type has been developed for biomedical image segmentation and thus, complies with the usual constraint of biomedical data, which is having small datasets of very high dimensionality. Indeed, the U-NET segmentation neural network is a model that has few parameters to train, making it possible to train this network with a fewer training examples. Moreover and in another embodiment, using data augmentation techniques on the training data can yield very good results with this architecture allowing to get more training examples from the same training sets.

In a further embodiment, the original image can be downsampled in order to make the image segmentation step less computationally expensive. As will be described further below and in one embodiment, some of the image analysis is performed at a tile level (which is a subsection of the image), using the semantic segmentation on a downsampled version of the image does not degrade the quality of the segmentation. This allows the use of downsampled image without degrading the quality of the segmentation. In one embodiment, to obtain the segmentation mask for the original full resolution image, process 100 simply needs to upscale the segmentation mask generated by the neural network.

At block 106, process 100 tiles ROI into a set of tiles. Tiling the image can include dividing the original image into smaller images that are easier to manage, called tiles. In one embodiment, the tiling operation is performed by applying a fixed grid to the whole-slide image, using a segmentation mask generated by a segmentation method, and selecting the tiles that contain tissue, or any other region of interest. In order to reduce the number of tiles to process even further, in one embodiment, additional or alternative selection methods can be used, such as random subsampling to keep only a given number of slides.

Tiling can be performed to increase the ability of preprocessing the images. For example, and in one embodiment, using a tiling method is helpful in histopathology analysis, due to the large size of the whole-slide image. More broadly, when working with specialized images, such as histopathology slides, or satellite imagery, or other types of large images, the resolution of the image sensor used in these fields can grow as quickly as the capacity of random-access memory associated with the sensor. With this increased image size, it is difficult to store batches of images, or sometimes even a single image, inside the random-access memory of a computer. This difficulty is compounded if trying to store these large images in specialized memory of a Graphics Processing Unit (GPU). This situation makes it computationally intractable to process an image slide, or any other image of similar size, in its entirety.

In one embodiment, tiling the image (or the image minus the background) addresses this challenge by dividing the original image (or the image minus the background), into smaller images that are easier to manage (i.e., tiles). In one embodiment, the tiling operation is performed by applying a fixed grid to the whole-slide image, using the segmentation mask generated by the segmentation method, and selecting the tiles that contain tissue, or any other kind of region of interest for the later classification process. In order to reduce the number of tiles to process even further, additional or alternative selection methods can be used, such as random subsampling to keep only a given number of slides. For example, and in one embodiment, the image (or the image minus the background) is divided into tiles of fixed size (e.g., each tile having a size of 224×224 pixels). A tile can be of any uniform size within a slide. Tile can be a square. For example, a tile can have about 10-500 μm width and/or depth. A tile can have about 20-1,000 pixels per side. The number of tiles generated depends on the size of the matter detected and can vary from a few hundred tiles to 50,000 or more tiles. In one embodiment, the number of tiles is limited to a fixed number that can be set based on at least the computation time and memory requirements (e.g., 10,000 tiles). In specific embodiments, tiles have about 224×224 pixels and a size of about 112 μm×112 μm. In these embodiments, digitalized whole slide image can have about 10,000 tiles per image.

In one embodiment, augmentations may be applied to each of the sets of tiles. In some embodiments, a first set of features is extracted from a first batch of augmented tiles. A second set of features is extracted from a second batch of augmented tiles. In some embodiments, the augmented tiles include zoomed in or rotated views, or views with color augmentations. For example, since orientation is not important in histology slides, the slides can be rotated at various degrees. The slides can also be enlarged or zoomed in. Contrastive loss between pairs of the first and second set of extracted features can be used in order to bring matching pairs of tiles closer and different pairs of tiles further apart. Contrastive loss can be applied in order to pay attention to positive pairs taken from the first and second set of features, rather than negative pairs.

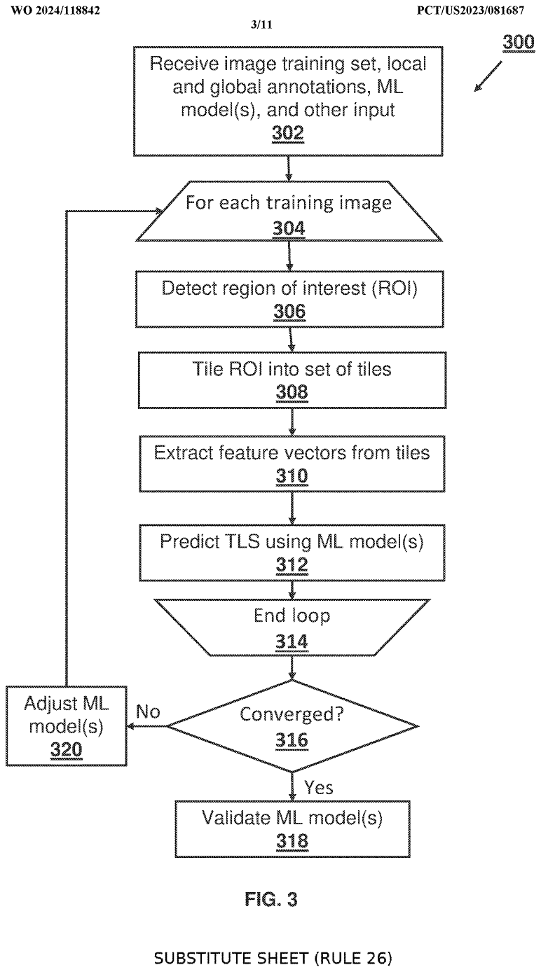

At block 108, process 100 extracts feature vectors from each tile. In one embodiment, the feature extraction is performed by ResNet50 neural network. In one embodiment, each of the features are extracted by applying a trained feature extractor that was trained with a machine learning algorithm using a training set of images. For example and in one embodiment, the machine learning algorithm is Momentum Contrast (MoCo) or Momentum Contrast v2 (MoCo v2). In one embodiment, the trained machine learning model is the machine learning model as trained in FIG. 3 described herein.

In some embodiments, a machine learning algorithm, e.g., MoCo or MoCo v2, extracts a plurality of feature vectors from each tile. The extracting of a plurality of feature vectors can be performed using a convolutional neural network, e.g., a ResNet50 neural network. The plurality of feature vectors can be any number, such as about 1,000, about 1,500, about 2,000, about 3,000, about 4,000, or more.

In some embodiments, a machine learning algorithm, e.g., MoCo or MoCo v2, extracts a plurality of feature vectors from the digital image and the extracting a plurality of feature vectors is performed using a first convolutional neural network. In one embodiment, process 100 can use any feature extraction neural network to extract the features, such as a ResNet based architecture (ResNet-50, ResNet-101, ResNetX etc.), Visual Geometry Group (VGG) neural network, Inception neural network, or a custom-made neural network specifically designed for the task. In some embodiments, non neural network feature extractors such as SIFT or CellProfiler can be used for extracting features. Moreover, the feature extraction neural network used can be a pre-trained one as these are trained on very large-scale datasets, and thus have an optimal generalization accuracy. In one embodiment, the first neural network comprises a 1D convolutional layer. In one embodiment, the 1D convolutional layer uses as input MoCo features and outputs a likelihood that the tile comprises a TLS.

In one embodiment, process 100 uses a ResNet-50 neural network as this neural network can provide well suited features for image analysis without requiring too much computing resources. For example and in one embodiment, the ResNet-50 can be used for histopathological image analysis. In this example, the ResNet-50 neural network relies on residual blocks that allow the neural network to be deeper and still improve its accuracy, as simple convolutional neural network architectures can get worst accuracies when the number of layers grows too large. In one embodiment, the weights of the ResNet-50 neural network can be the weights used for the feature extraction are from a pre-training on the dataset ImageNet, since this dataset is a really general-purpose image dataset. In one embodiment, using a neural network pre-trained on a large independent image data set provides good features independently of the kind of images, even in the case where the input images are specialized, as is for histopathological images (or other types of images). In this embodiment, process 100 uses ResNet-50 convolutional neural network to extract 2,048 features per tile. If process 100 extracts 10,000 tiles, for example, process 200 generates a matrix of 2,048×10,000. Furthermore, if process 200 is being executed with a number of images as input then process 100 generates a tensor with dimensions of: number of images×number of features/tile×number of tiles.

Process 100, in one embodiment and in order to extract features for a given slide, processes each of the selected tiles goes through the ResNet-50 neural network outputting the feature vector for that tile. In this embodiment, the feature vector can be a vector of dimensional 2048 or another size. In addition, process 100 can apply an autoencoder to the feature vectors to further provide dimensionality reduction (e.g., reducing the dimensions of the feature vectors to 256 or another dimensional). In one embodiment, the autoencoder can be used when the machine learning model may be susceptible to over fitting. For example and in one embodiment, process 100 can reduce the length of a 2,048 feature vector down to a 512 length feature vector. In this example, the process 100 uses the autoencoder, which includes a single hidden-layer architecture (of 512 neurons). This prevents the model from over-fitting by finding several singular features in the training dataset and also reduces computation time and required memory. In one embodiment, the classification model is trained on a small subset of the image tiles, e.g., trained on 200 tiles randomly selected from each slide (out of a total of 411,400 tiles).

Process 100 can optionally perform a zero-padding operation on the feature vectors, in order to derive a minimal number of features. In this embodiment, process 100 can perform a zero-padding to add feature vectors to the set of feature vectors for the image if the number of feature vectors is below a minimal number of feature vectors. In this embodiment, each zero-padded feature vector has null values.

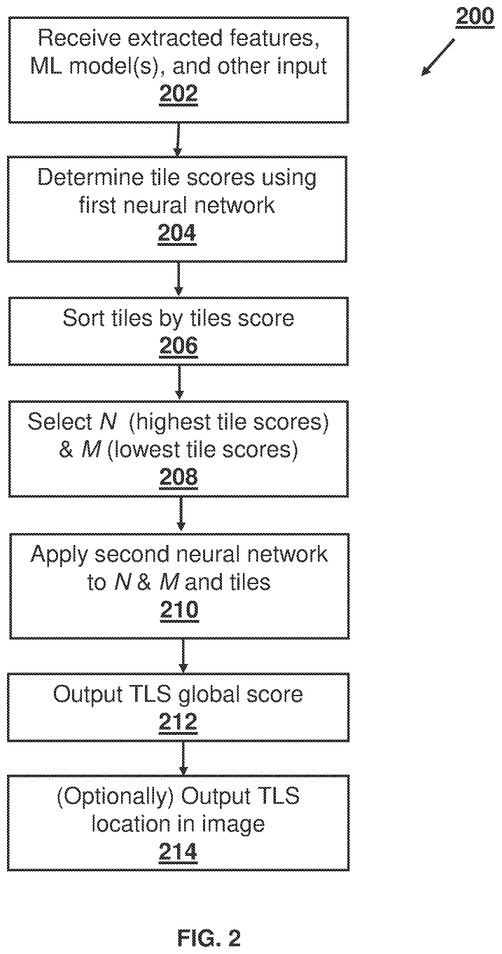

At block 110, process 100 classifies the image for the TLS status using extracted feature vectors and machine learning models. The computer-implemented method can process all tiles within the ROI for extracting feature vectors and/or classifying. In some embodiments, the computer-implemented method further comprises selecting a subset of tiles for application to the machine learning model. In some embodiments, the subset of tiles is selected by random sampling. In this embodiment, process 100 can output that the digital image is TLS positive or TLS negative, where this designation is a global classification of the digital image. Alternatively, process 100 can determine which tiles are TLS positive, based on a tile score of that tile. In one embodiment, the TLS image classification is further discussed in FIG. 2 below.

In some embodiments, the classifying step, such as block 110 in process 100, comprises:
applying a first neural network to said one or more of the plurality of feature vectors, wherein said first neural network assigns a tile score to each tile of said set of tiles based on said one or more of the plurality of feature vectors, wherein the tile score represents a likelihood that said tile comprises a TLS; and
applying a second neural network to said each tile, wherein said second neural network aggregates subsets of said tile score of said set of tiles and determines the TLS status in the histology image, wherein:
said first neural network is trained using a training set of histology images comprising known local annotations of a presence or absence of TLSs at tile level; and
said first neural network and second neural network are trained using a training set of histology images comprising known global annotations of a presence or absence of a TLS at histology image level.

FIG. 2 illustrates an example flow diagram for a process 200 that of classifies an image for the TLS status and optionally predicts a TLS location within the image according to embodiments of the present disclosure. In one embodiment, process 100 executes FIG. 2 to classify an image for a TLS status.

At block 202, process 200 receives features extracted from each tile, machine learning model(s), and optionally other input. In one embodiment, the extracted features are the extracted features determined in block 108 above. At block 204, process 200 determines tile scores for each tile based on the extracted feature vectors, using a first neural network that was trained using a training image set with local annotations. In some embodiments, the first neural network is a convolutional 1D layer. In some embodiments, the tile score represents a likelihood that said tile comprises a TLS. In one embodiment, the tile scores are expressed in a continuous range. For example and in one embodiment, process 200 determines tile scores for each tile that are any number between 0 and 1, with score 0 indicating the lowest likelihood that the tile comprises a TLS, and score 1 indicating the highest likelihood that the tile comprises a TLS. In one embodiment, process 200 reduces each of the feature vectors to one or more scores using a connected neural network. In one embodiment, process 200 can reduce the feature vector to a single score using a fully connected neural network, or to multiple scores representing various characteristics of the tile using one fully connected neural network outputting various scores or a plurality of fully connected neural networks, each outputting a different score. These scores, associated with one tile, are sorted and a subset of the tiles is selected for the image classification.

For example and in one embodiment, process 200 can use a convolutional 1D layer to create a score for each tile. In this example, as described above with feature vectors of 2,048 length, this convolutional layer performs a weighted sum between the 2,048 features of the tile to obtain this score, where weights of this sum are learned by the model. Furthermore, because the convolutional 1D layer is unbiased, the zero-padding tiles have a score of zero and thus a reference for a totally uninformative tile. Process 200 picks the highest N scores and lowest M scores and uses them as input for the classifying described below. This architecture ensures which tiles are used to make the predictions.

In one embodiment, process 200 uses the tile score vector as input to a dense multilayer neural network to provide the desired classification (e.g., the TLS status of the image). This classification can be any task that associates labels to the data given as input to the classifier. In one embodiment, using a trained classifier for the digital image inputs since said input data is derived by the whole pipeline, the classifier is thus capable to label the histopathology image given as input without needing to process the full image, which can be computationally prohibitive. For example and in one embodiment, the labels can be a label of any kind, such as: binary values representing prognosis of a given pathology; numeric labels representing a score, a probability, or a prediction of a physical quantity, such as survival prediction or response to treatment prediction; and/or a scalar label as described previously or a vector, matrix or tensor of such labels representing structured information. For example and in one embodiment, process 200 can output a TLS positive or TLS negative status, indicating whether the digital image is predicted to include or not include TLS. In one embodiment, process 200 uses a multi-layer perceptron (MLP) with two fully connected layers of 200 and 100 neurons with sigmoid activation. In this embodiment, the MLP is used as a core of the predictive algorithm that transforms the tile scores to label(s). While in one embodiment, process 200 predicts a single label for the image (e.g., a probability score), in alternate embodiments, process 200 can predict which of the tiles can include TLS. In this embodiment, process 200 can determine that a certain score is a threshold for determining the presence of TLS in a tile.

The histology image can be classified based on at least a set of tile scores that are derived from the image tile feature vectors generated from the neural network. At block 204, process 200 computes a tile score for each tile using the associated feature vector for that tile. For example and in one embodiment, process 200 can use a convolutional 1D layer to create a score for each tile. In the example described above with feature vectors of 2,048 length, this convolutional layer performs a weighted sum between all 2,048 features of the tile to obtain this score, where weights of this sum are learned by the model. Furthermore, because the convolutional 1D layer is unbiased, the zero-padding tiles have a score of zero and thus a reference for a totally uninformative tile.

At block 206, process 200 sorts tiles by tile score. In one embodiment, process 200 sorts the tile set to determine the top N and/or bottom M scores for block 206.

At block 208, process 200 selects highest tile scores (N) and lowest tile scores (M). In one embodiment, process 200 selects a subset of tiles which is used for the classification step later on. In one embodiment, this subset of tiles can be tiles with the top N highest scores and the bottom M lowest scores, the top N highest scores, the bottom M lowest scores, and/or any weighted combination of the scores. In one embodiment, the ranges of values for N and/or M can be the same or different. In addition, the N and/or M ranges can be a static numerical range (e.g., 10, 20, 100, or some other number), adapted to a range, a percentage, a label (e.g., small, large, or some other label), set via a user interface component (slider, user input, and/or another type of user interface component), and/or some other value. In one embodiment, process 200 additionally concatenates these scores into an image score vector that can be taken as input for the image classification.

In some embodiments, the classifying step further comprises:
sorting said set of tiles by
   picking tiles comprising highest TLS tile scores, and
   picking tiles comprising lowest TLS tile scores.

In one embodiment, when studying histology whole-slide images (or slides), a single patient can be associated with multiple slides, taken with various stainings, at various locations of the same sample, from multiple organs, or at various time points. In this embodiment, the slides from a single patient can be aggregated in multiple ways. In one embodiment, process 200 can concatenate the slides, in order to form a larger slide that will be processed in the same or similar way as a normal one (segmentation, tiling, feature extraction and classification).

In another embodiment, a slide may not contain enough useful tissue to extract as tiles on which to apply the feature extraction step and thus to feed the classifier with features. In this case, the input of the classifier is zero padded, meaning that for every tile lacking, a feature consisting of zeros is added to the real features computed by the feature extractor.

At block 210, process 200 applies a second neural network to tiles using the tile scores. In one embodiment, the second neural network is a Multi-Layer Perception model. In one embodiment, process 200 uses a subset of the tiles, namely the tile scores N and M. At block 212, process 200 determines a TLS global score for the histology image using the result of the second neural network applied to the tile scores. In one embodiment, the TLS global score can be a positive occurrence of TLS or a negative occurrence of TLS. In this embodiment, the number of tiles for the image can be on the order of 10,000 tiles. In further embodiment, there can be more or less number of tiles for the image. In one embodiment, and to reduce the computational complexity, the classification system samples the tiles to reduce the number of tiles that are used in the neural network computations. In one embodiment, the classification system samples the tiles randomly or some other type of sampling mechanism. For example and in one embodiment, the classification system randomly samples the tiles to reduce the number of tiles from on the order of 10,000 tiles to on the order of a few thousand tiles (e.g., 3000 tiles).

In some embodiments, the computer-implemented method provided herein further comprises detecting one or more locations in which a TLS is present in said histology image.

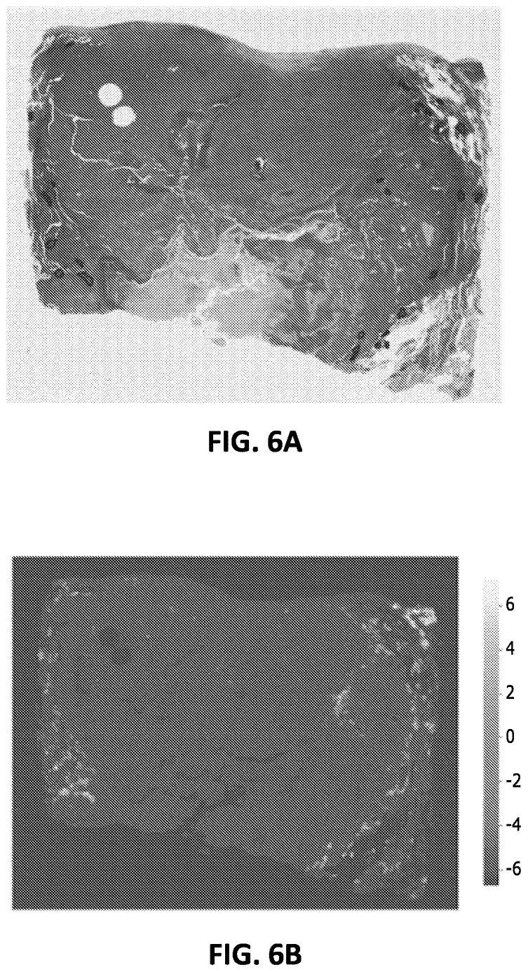

For example, process 200 can optionally continue to block 214, where process 200 outputs predicted TLS location within the histology image. In some embodiments, at block 214, process 200 overlays tile scores assigned to each tile onto the histology image, such that the areas assigned high and low tile scores are visualized. For example, as shown in FIG. 6B, process 200 can detect TLS locations within a histology image. Areas are color coded according to the assigned tile scores, with brighter areas corresponding to higher tile scores indicative of higher likelihood of a TLS. The detection of TLS locations by corresponds with manual annotations of TLS locations by pathologist in FIG. 6A, wherein areas marked in blue lines indicate TLS locations.

In some embodiments, the computer-implemented method provided herein comprises:
repeating all the steps of:
   receiving a digitalized histology image of a sample obtained from the subject;
   tiling the histology image into a set of tiles;
   extracting a plurality of feature vectors from each of said tiles, wherein each feature of said one or more feature vectors represents local descriptors of the tile; and
   classifying the histology image for a TLS status using at least the plurality of feature vectors and a classification model that is trained with a imaging training set having known TLS annotations
for each histology image in a plurality of histology images, and
processing the TLS status of the plurality of histology images, thereby detecting the presence or absence TLS in a subject.

The machine learning models of the computer-implemented methods provided herein can be trained and validated. In specific embodiments, the first neural network is trained using a training set of histology images comprising known local annotations of a presence or absence of TLSs at tile level; and the first neural network and second neural network are trained using a training set of histology images comprising known global annotations of a presence or absence of a TLS at histology image level.

In one embodiment, the model(s) used in FIGS. 1 and 2 can be trained and validated using a training set of images. In this embodiment, process 300 trains the one dimensional convolutional neural network producing the scores and the MLP classification model using input labels of the training set of images. In this embodiment, a process iteratively trains the model(s) by computing the score sets for the training image, predicting the labels, determining differences between the predicted labels and the input labels, optimizing the model(s) based on the difference (e.g., computing new weights for the model(s)), until the differences are within a threshold. In one embodiment, process 300 trains the model to predict a single label for the image (e.g., a tile score). In alternate embodiments, the process can be trained to predict multiple global labels for the image. In one embodiment, the process can be trained to perform a multi-task learning environment to predict multiple global labels. For example and in one embodiment, the machine learning model (e.g., the MLP and/or other model(s) described elsewhere) can be trained to predict multiple labels at once in the multi-task learning environment using the resulting feature vector.

FIG. 3 illustrates a flow diagram of one embodiment of a process 300 to train and validate the first and second neural networks for detecting TLS status in histology images or subjects. In one embodiment, the neural networks includes one or more separate models used for the classification process described in FIGS. 1 and 2. In FIG. 3, process 300 begins by receiving a training set of histology images, local and global annotations, machine learning model(s), and optionally other input at block 302. In one embodiment, process 300 receives images, local and global annotations, the first and second neural networks used in FIGS. 1 and 2 above, and optionally other input as described in FIG. 1. In one embodiment, the first neural network is a 1D convolutional layer. In one embodiment, the 1D convolutional layer uses MoCo features as input and outputs a likelihood that the tile comprises a TLS. In one embodiment, the second neural network is a Multi-Layer Perception model. In one embodiment, the first neural network is trained using a training set of histology images comprising known local annotations of a presence or absence of TLSs at tile level; and the first neural network and second neural network are trained using a training set of histology images comprising known global annotations of a presence or absence of a TLS at histology image level. Process 300 performs a processing loop (blocks 304-314) for each training image to generate a set of feature vectors from tiles and predict the TLS status of the images. At block 306, process 300 detects region of interest (ROI) in the training image. In one embodiment, process 300 detects the ROI as described in FIG. 1, block 104 above. At block 308, process 300 tiles the ROI into a set of tiles. In one embodiment, process 300 tiles the ROI as described in FIG. 1, block 106 above. At block 310, process 300 extracts the feature vectors from the tiles. In one embodiment, process 300 extracts the feature vector as described in block 108, FIG. 1 above. For example and in one embodiment, process 300 uses a ResNet-50 convolutional neural network to determine the feature vector for each tile of a tiled segment image as described in FIG. 1 above. In one embodiment, process 300 generates a set of feature vectors for the training image. In addition, process 300 can perform data augmentation during the training of the method to improve the generalization error. This data augmentation can be done by applying various transformations on the tiles such as rotations, translations, flipping, cropping, blurring, adding noise to the image, modifying the intensity of particular colors, or changing the contrast. In one embodiment, process 300 determines the tile score using the first neural network as described in block 204, FIG. 2 above. In one embodiment, process 300 continues to sorting tiles by tile score as described in block 206, FIG. 2 above. In one embodiment, process 300 continues to selecting N (highest tile scores) and M (lowest tile scores) as described in block 208, FIG. 2 above. In one embodiment, process 300 continues to applying the second neural network to N, M, and tiles, as described in block 210, FIG. 2 above. At block 312, process 300 predicts the TLS status (e.g., presence, amount, or location of TLSs) in each histology image using the extracted feature vectors and the machine learning models. In one embodiment, process 300 predicts the TLS status (e.g., presence, amount, or location of TLSs) in each histology image using the extracted feature vectors and the machine learning models as described in block 110, FIG. 1 above, output TLS global score as described in block 212, FIG. 2 above, and/or output TLS location in the histology image as described in block 214, FIG. 2 above. The process loop ends at 314.

In order to determine the adequacy of the training, at block 316, process 300 evaluates whether the prediction of the TLS status by the machine learning model and the global and local annotations of the TLS status in the training data set have converged. If they have converged, at block 318, process 300 validates the machine learning model. If they have not converged, at block 320, process 300 adjusts the machine learning model and reperforms the processing loop (blocks 304-314).

In FIG. 3, process 300 trained a machine learning model that is used to classify images. The accuracy of the machine learning model can be assessed by validating the machine learning model using the training set of images as inputs and computing one or more labels. A validation process can be conducted by receiving a validation image set and the trained machine learning model; and processing the validation image set. In one embodiment, the validation image set is the same as the training set. In another embodiment, the validation set can be different from the training image set. For example and in embodiment, an image set that has been labeled for a particular type of image (e.g., histology images) can have some image selected for use in training the models and other images from this set be used for validating the trained models. In one embodiment, the model is a machine learning model, such as a MLP model described above.

In some embodiments of the computer-implemented methods provided herein, each tile comprises a plurality of pixels. In some embodiments, the method further comprises detecting pixel-based segmentation of a TLS within a tile. Such determination step can comprise applying a machine learning model to each tile of said set of tiles, wherein said model is trained using a training set of tiles comprising known pixel-based TLS segmentation mask within a tile. “TLS segmentation” or “Segmentation of a TLS” as used herein refers to localization of TLS in a given area. TLS segmentation in a given area (e.g., tile, histology image) can be expressed by the presence or absence of TLS in unit areas (e.g., pixels) tiling the given area. In some embodiments, said model assigns a pixel score to each pixel of each tile of said set of tiles and determines said pixel-based segmentation of a TLS within a tile, wherein the pixel score represents a likelihood that said pixel comprises a TLS.

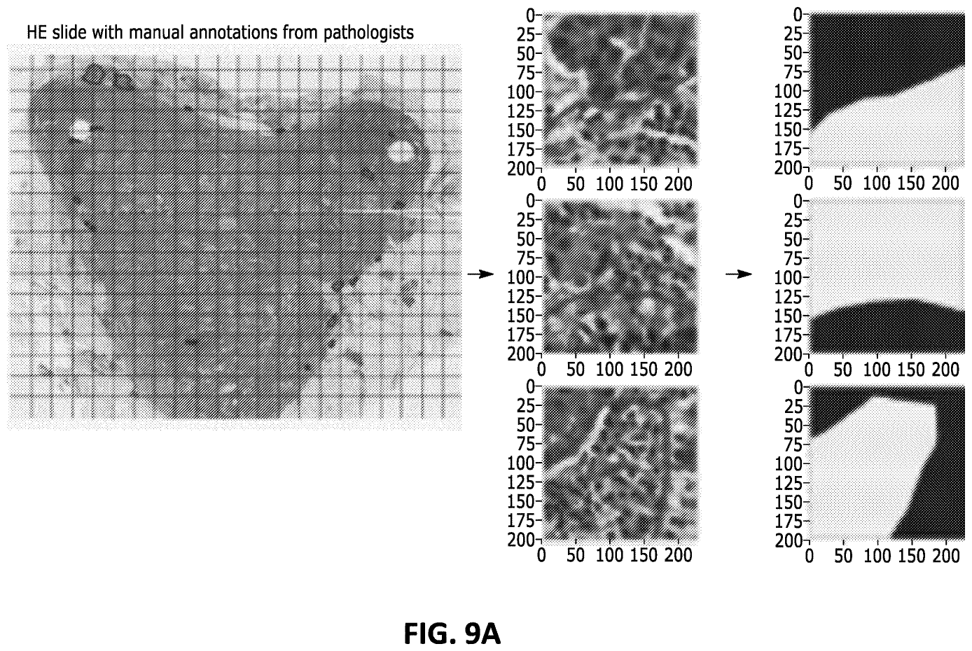

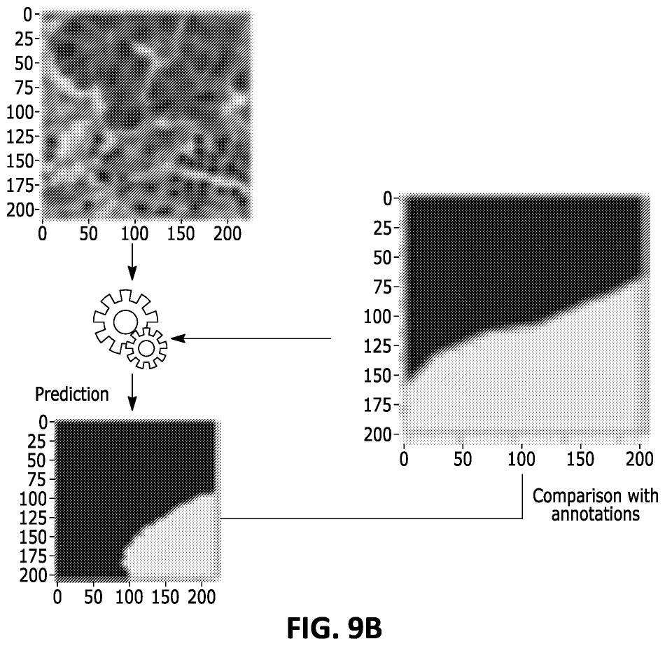

For example, as shown in FIG. 9A, the training set of images can have local manual annotations of tiles and their segmentation masks from an H&E stained histology image based on manual annotations by pathologists of TLS locations as marked by blue lines. As shown in FIG. 9B, a machine learning model can be trained to predict the TLS segmentation by processing the training set of images with known pixel-based segmentation masks and comparing the output with annotations.

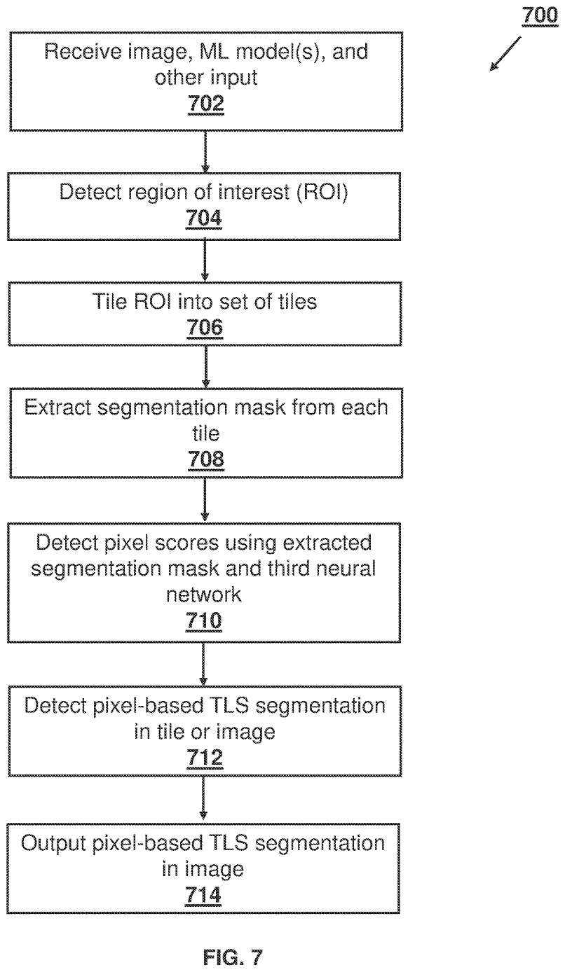

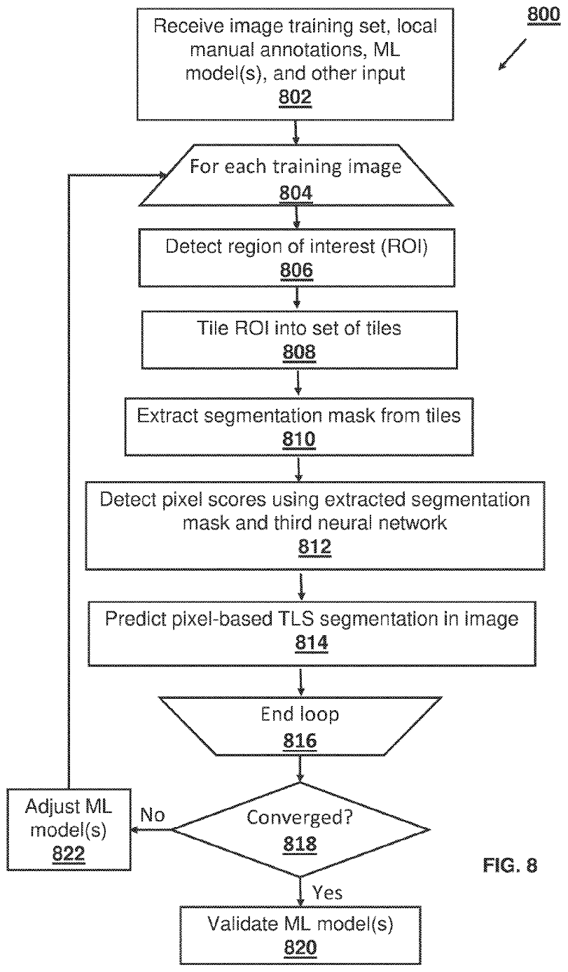

FIG. 7 illustrates an example flow diagram for a process 700 that detects the pixel-based TLS segmentation in an image using machine learning model, according to embodiments of the present disclosure. At block 702, process 700 receives an image, one or more machine learning (ML) models, and optionally other input. In some embodiments, the image is a digitalized histology image of a sample obtained from the subject, such as a digitalized WSI. At block 704, process 700 detects a region of interest (ROI) within the image, as described in block 104, FIG. 1. At block 706, process 700 tiles ROI into set of tiles, as described in block 106, FIG. 1. At block 708, process 700 extracts pixel-based segmentation mask from each tile. At block 710, process 700 determines pixel scores for each pixel within a tile. At block 712, process 700 detects pixel-based segmentation in the tile or image using the third neural network. In some embodiments, determination of pixel scores and/or detection of pixel-based segmentation in the tile or image is performed using a third neural network, trained using a training set of images with local annotations of pixel-based segmentation mask, as described herein and for example in FIG. 8. In one embodiment, the third neural network is a semantic segmentation neural network, such as a U-NET semantic segmentation neural network, or another type of semantic segmentation neural network. In one embodiment, a semantic segmentation neural network can be used that does not depend on a particular distribution in the intensity histogram. Moreover, using such a neural network allows the image segmentation to take into account multichannel images such as Red-Green-Blue (RGB) images. Thus, the segmentation does not just rely on the histogram of pixel intensity but can take advantage of the semantics of the image. At block 714, process 700 outputs pixel-based TLS segmentation in the histology image.

FIG. 8 illustrates a flow diagram of one embodiment of a process 800 to train and validate the machine learning models for detecting TLS segmentation in histology images or subjects. In one embodiment, the machine learning models to be trained include one or more separate models used for the segmentation detection process described in FIG. 7. In one embodiment, the machine learning model to be trained is the third neural network described in FIG. 7. In FIG. 8, process 800 begins by receiving a training set of histology images, local and global annotations, machine learning model(s), and optionally other input at block 802. In one embodiment, process 800 receives images, local manual annotations, the third neural network used in FIG. 7, and optionally other input as described in FIG. 7. In one embodiment, the third neural network is a semantic segmentation neural network, such as a U-NET semantic segmentation neural network. In one embodiment, the third neural network is trained using a training set of histology images comprising known local manual annotations of TLS locations (i.e., TLS segmentations) in the image, as shown in FIG. 9A. Process 800 performs a processing loop (blocks 804-816) for each training image to generate a set of segmentation masks from tiles and predict the pixel-based TLS segmentation of the images. At block 806, process 800 detects region of interest (ROI) in the training image. In one embodiment, process 800 detects the ROI as described in FIG. 7, block 704 above. At block 808, process 800 tiles the ROI into a set of tiles. In one embodiment, process 800 tiles the ROI as described in FIG. 7, block 706 above. At block 810, process 800 extracts the segmentation masks from the tiles. In one embodiment, process 800 extracts the segmentation masks as described in block 708, FIG. 7 above. In one embodiment, process 800 generates a set of segmentation masks for the training image. At block 812, process 800 detects pixel scores using extracted segmentation mask and the third neural network. In one embodiment, process 800 detects pixel scores using extracted segmentation mask and the third neural network as described in block 712, FIG. 7. At block 814, process 800 predicts pixel-based TLS segmentation in the image. In one embodiment, process 800 predicts pixel-based TLS segmentation in the image as described in block 714, FIG. 7. The process loop ends at 816.

In order to determine the adequacy of the training, at block 818, process 800 evaluates whether the prediction of the TLS segmentation by the machine learning model and the local manual annotations of the TLS status in the training data set have converged. If they have converged, at block 820, process 800 validates the machine learning model. If they have not converged, at block 822, process 800 adjusts the machine learning model and reperforms the processing loop (blocks 804-816). A validation process can be conducted by receiving a validation image set and the trained machine learning model; and processing the validation image set, as described above.

In some embodiments, the trained feature extractor can be achieved after training for a certain number of epochs. In some embodiments, training is performed until precision is at or near 1 (or 100%), until the AUC is at or near 1 (or 100%), or until the loss is near zero. In some embodiments, during training of a feature extractor with contrastive loss one may not have access to an abundance of helpful metrics. Thus, one can monitor one of the available metrics of downstream tasks, like AUC, to see how the feature extractor is performing. In one example, a feature extractor that is trained at a certain epoch can be used to train a downstream weakly supervised task in order to evaluate performance. If additional training could result in improved downstream performance, such additional training may be warranted.

In some embodiments of the computer-implemented methods provided herein, said sample is a cancer. The methods provided herein can be used for a cancer samples of heterologous primary site, stage, pathological type, subject profile, or clinical/treatment course. The methods provided herein can also be used for cancer samples of a specific primary site, state, pathological type, subject profile, or clinical/treatment course.

For example, the methods provided herein can be used for any cancer that originated in any organ or tissue in the body. In some embodiments, the cancer is selected from the group consisting of lung cancer, sarcoma, bladder cancer, colorectal cancer, ovarian cancer, pancreatic cancer, and melanoma. The cancer sample can be derived from a subject at any time relative to a diagnosis of the cancer. For example, the sample can be obtained from the subject before or after the pathological diagnosis of the cancer. The sample can be obtained from the subject before or after a treatment (e.g., immunotherapy, chemotherapy, surgery, radiation) of the cancer.

In some embodiments of the computer-implemented method provided herein, the training set of tiles and/or training set of histology images are digitalized images of histology sections of cancers of heterologous origins (e.g., lung cancer, sarcoma, bladder cancer, colorectal cancer, ovarian cancer, pancreatic cancer, melanoma).

In some embodiments, the histology image is a digitalized whole slide image (WSI). In some embodiments, the digitalized histology image is that of the histologic section of the sample that has been stained with a dye to visualize the underlying tissue structure. The dye can be hematoxylin and eosin (H&E). Other common stains that can be used to visualize tissue structures in the input image include, for example, Masson's trichome stain, Periodic Acid Schiff stain, Prussian Blue stain, Gomori trichome stain, Alcian Blue stain, or Ziehl Neelsen stain.

In some embodiments, the machine learning model is a Deep Multiple Instance Learning model. In some embodiments, the machine learning model is a Weldon model. In some embodiments, the machine learning model is applied to the entire group of tiles. In some embodiments, the machine learning model is applied to a subset of tiles. The training images can include digital images of histologic sections derived from a number of control subjects. In some cases, the training images lack local annotations. The training images can include images associated with one or more global label(s) indicative of one or more TLS feature(s) of the patient from whom the sample is derived.

The machine learning models described herein can identify TLS locations in a histology image. TLS locations can be identified by, for example, selection of a cohort of tiles having highest M and lowest N scores. The highest and lowest cohorts of tiles identified by the model as having the best correlation with the presence or absence of TLS can be analyzed by a pathologist to determine TLS features within the tiles. For example, in some embodiments, TLS features can be determined by analysis of the cohort of tiles having M scores in the top 20%, e.g., top 15%, top 10%, top 5%, top 2%, top 1%, etc., and/or having N scores in the bottom 20%, e.g., bottom 15%, bottom 10%, bottom 5%, bottom 2%, bottom 1%, etc. of all of the tiles assessed by the model.

TLS features include one or more of the following, and combinations thereof:
a. structural features found in secondary lymphoid organs (SLOs) (e.g., lymph nodes, tonsils, spleen, Peyer's patches, or mucosa associated lymphoid tissue), such as lymphoid follicles including a dense cellular aggregate resembling germinal centers in the SLOs;
b. nonencapsulated, nonlymphoid tissues such as Peyer's patches and pre-existing lymphoid follicles;
c. lymphocyte aggregates;
d. B-cell follicles with actively replicating B-cell germinal centers surrounded by a T-cell region;
e. mature dendritic cells in T cell zones and/or follicular dendritic cells in B cell zones (i.e., follicles); and
f. a heterologous population of cells and structures, including at least one of discrete B cell zones, T cell zones, marginal zones with activated macrophage and dendritic cells, reticular fibroblast cell (RFC) networks (or RFC-like stromal networks), vasculature permissive to immune cell extravasation (e.g., high endothelial venules, which are blood vessels expressing peripheral node addressins (PNAd) and specialized in the extravasation of circulating immune cells), dendritic cell-lysosomal associated membrane protein (DC-LAMP), and dendritic cells;

In some embodiments, a histology image weighing toward presence of TLSs contains one or more, two or more, three or more, four or more, five or more, or six of the foregoing TLS features. In some embodiments, the histology image is a whole slide image. In other embodiments, the histology image is a section of a whole slide image, e.g., a tile derived from a whole slide image.

The presence or absence of TLS features described herein can be determined in an image obtained from a tissue of the subject. The image can be, for example, a whole slide image (WSI), or a portion thereof, e.g., a tile derived from a WSJ. In exemplary embodiments, the tissue is derived from a biopsy, e.g., cancer biopsy, obtained from the subject. Suitable sources of tissue for a biopsy are known in the art, and include without limitation, tissue samples obtained from a needle biopsy, an endoscopic biopsy, or a surgical biopsy. In exemplary embodiments, the image is derived from a thoracentesis biopsy, a thoracoscopy biopsy, a thoracotomy biopsy, a paracentesis biopsy, a laparoscopy biopsy, or a laparotomy biopsy.

Tissue sections can be processed for image analysis using any suitable methods and stains for histopathology analysis. For example, the tissue sections can be stained with hematoxylin and eosin, alkaline phosphatase, methylene blue, Hoechst stain, and/or 4′, 6-diamidino-2-phenylindole (DAPI).

The classification algorithm can compute a TLS score for the subject, which is indicative of the TLS status. The TLS score can be classification, e.g., presence or absence of TLS. The TLS score can also be a continuous probability score. The continuous TLS score of a subject, e.g., a cancer subject, can be plotted against the scores obtained from a plurality of subjects of known TLS status to determine the TLS status of the test subject.

### II. Computer System and Machine Readable Medium

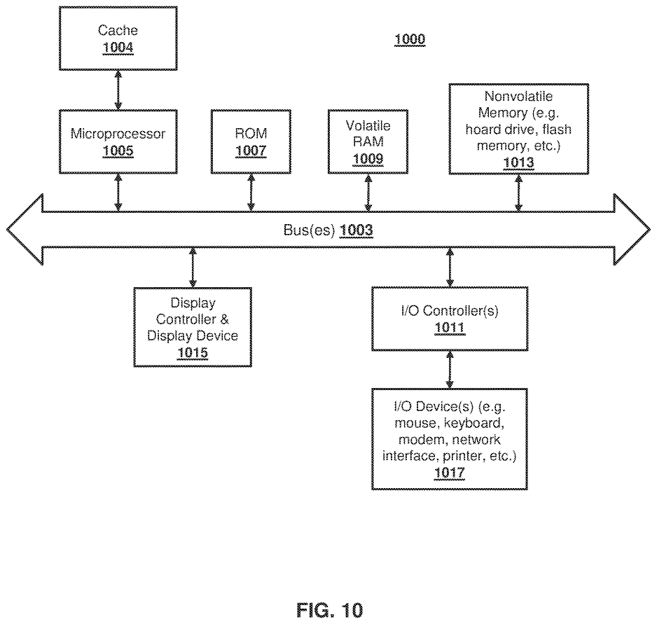

As shown in FIG. 10, the computer system 1000, which is a form of a data processing system, includes a bus 1003 which is coupled to a microprocessor(s) 1005 and a ROM (Read Only Memory) 1007 and volatile RAM 1009 and a non-volatile memory 1013. The microprocessor 1005 may include one or more CPU(s), GPU(s), a specialized processor, and/or a combination thereof. The microprocessor 1005 may be in communication with a cache 1004, and may retrieve the instructions from the memories 1007, 1009, 1013 and execute the instructions to perform operations described above. The bus 1003 interconnects these various components together and also interconnects these components 1005, 1007, 1009, and 1013 to a display controller and display device 1015 and to peripheral devices such as input/output (I/O) devices 1011 which may be mice, keyboards, modems, network interfaces, printers and other devices which are well known in the art. Typically, the input/output devices 1011 are coupled to the system through input/output controllers 1017. The volatile RAM (Random Access Memory) 1009 is typically implemented as dynamic RAM (DRAM), which requires power continually in order to refresh or maintain the data in the memory.

The nonvolatile memory 1013 can be, for example, a magnetic hard drive or a magnetic optical drive or an optical drive or a DVD RAM or a flash memory or other types of memory systems, which maintain data (e.g. large amounts of data) even after power is removed from the system. Typically, the nonvolatile memory 1013 will also be a random access memory although this is not required. While FIG. 10 shows that the nonvolatile memory 1013 is a local device coupled directly to the rest of the components in the data processing system, it will be appreciated that the present invention may utilize a nonvolatile memory which is remote from the system, such as a network storage device which is coupled to the data processing system through a network interface such as a modem, an Ethernet interface or a wireless network. The bus 1003 may include one or more buses connected to each other through various bridges, controllers and/or adapters as is well known in the art.

Portions of what was described above may be implemented with logic circuitry such as a dedicated logic circuit or with a microcontroller or other form of processing core that executes program code instructions. Thus processes taught by the discussion above may be performed with program code such as machine-executable instructions that cause a machine that executes these instructions to perform certain functions. In this context, a “machine” may be a machine that converts intermediate form (or “abstract”) instructions into processor specific instructions (e.g., an abstract execution environment such as a “virtual machine” (e.g., a Java Virtual Machine), an interpreter, a Common Language Runtime, a high-level language virtual machine, etc.), and/or, electronic circuitry disposed on a semiconductor chip (e.g., “logic circuitry” implemented with transistors) designed to execute instructions such as a general-purpose processor and/or a special-purpose processor. Processes taught by the discussion above may also be performed by (in the alternative to a machine or in combination with a machine) electronic circuitry designed to perform the processes (or a portion thereof) without the execution of program code.

The present invention also relates to an apparatus for performing the operations described herein. This apparatus may be specially constructed for the required purpose, or it may comprise a general-purpose computer selectively activated or reconfigured by a computer program stored in the computer. Such a computer program may be stored in a computer readable storage medium, such as, but is not limited to, any type of disk including floppy disks, optical disks, CD-ROMs, and magnetic-optical disks, read-only memories (ROMs), RAMs, EPROMs, EEPROMs, magnetic or optical cards, or any type of media suitable for storing electronic instructions, and each coupled to a computer system bus.

A machine readable medium includes any mechanism for storing or transmitting information in a form readable by a machine (e.g., a computer). For example, a machine readable medium includes read only memory (“ROM”); random access memory (“RAM”); magnetic disk storage media; optical storage media; flash memory devices; etc.

An article of manufacture may be used to store program code. An article of manufacture that stores program code may be embodied as, but is not limited to, one or more memories (e.g., one or more flash memories, random access memories (static, dynamic or other)), optical disks, CD-ROMs, DVD ROMs, EPROMs, EEPROMs, magnetic or optical cards or other type of machine-readable media suitable for storing electronic instructions. Program code may also be downloaded from a remote computer (e.g., a server) to a requesting computer (e.g., a client) by way of data signals embodied in a propagation medium (e.g., via a communication link (e.g., a network connection)).

The preceding detailed descriptions are presented in terms of algorithms and symbolic representations of operations on data bits within a computer memory. These algorithmic descriptions and representations are the tools used by those skilled in the data processing arts to most effectively convey the substance of their work to others skilled in the art. An algorithm is here, and generally, conceived to be a self-consistent sequence of operations leading to a desired result. The operations are those requiring physical manipulations of physical quantities. Usually, though not necessarily, these quantities take the form of electrical or magnetic signals capable of being stored, transferred, combined, compared, and otherwise manipulated. It has proven convenient at times, principally for reasons of common usage, to refer to these signals as bits, values, elements, symbols, characters, terms, numbers, or the like.

It should be kept in mind, however, that all of these and similar terms are to be associated with the appropriate physical quantities and are merely convenient labels applied to these quantities. Unless specifically stated otherwise as apparent from the above discussion, it is appreciated that throughout the description, discussions utilizing terms such as “segmenting,” “tiling,” “receiving,” “computing,” “extracting,” “processing,” “applying,” “augmenting,” “normalizing,” “pre-training,” “sorting,” “selecting,” “aggregating,” “sorting,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

The processes and displays presented herein are not inherently related to any particular computer or other apparatus. Various general-purpose systems may be used with programs in accordance with the teachings herein, or it may prove convenient to construct a more specialized apparatus to perform the operations described. The required structure for a variety of these systems will be evident from the description herein. In addition, the present invention is not described with reference to any particular programming language. It will be appreciated that a variety of programming languages may be used to implement the teachings of the invention as described herein.

### III. Products

In some aspects, provided herein is a product capable of detecting the TLS status (e.g., presence, amount or location of TLSs) in a subject, a histology image, a tile within the histology image, or a pixel within the tile. In some aspects, the product is attached to a scanner. In some aspects, the scanner is capable of scanning pathology slides, e.g., H&E slides. In some aspects, the product is particularly useful for health care facilities, clinics, or providers, including those without expertise for cancer pathology, including making diagnosis or prognosis of cancer (e.g., lung cancer, sarcoma, bladder cancer, colorectal cancer, ovarian cancer, pancreatic cancer, and melanoma) or diagnosis of TLS. In some aspects, the product is useful for identifying personalized medicine or targeted therapy options for cancer in a subject.

### IV. General Considerations

The articles “a” and “an” are used herein to refer to one or to more than one (i.e., to at least one) of the grammatical object of the article. By way of example, “an element” means one element or more than one element, e.g., a plurality of elements.

The term “including” is used herein to mean, and is used interchangeably with, the phrase “including but not limited to.” The term “including” does not necessarily imply that additional elements beyond those recited must be present.

The term “about” or “approximately” when referring to a number or a numerical range means that the number or numerical range referred to is an approximation within experimental variability (or within statistical experimental error), and, thus, the number or numerical range may vary from, for example, between 1% and 20% of the stated number or numerical range. In some aspects, “about” indicates a value within 20% of the stated value. In more preferred aspects, “about” indicates a value within 10% of the stated value. In even more preferred aspects, “about” indicates a value within 1% of the stated value.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the following specification and claims are approximations that may vary depending on the desired properties sought to be obtained in aspects of the present invention. Notwithstanding that the numerical ranges and parameters setting forth the broad scope of the invention are approximations, the numerical values set forth in the specific examples are reported as precisely as possible. Any numerical values; however, inherently contain certain errors necessarily resulting from error found in their respective measurements.

The term “at least” prior to a number or series of numbers is understood to include the number adjacent to the term “at least”, and all subsequent numbers or integers that could logically be included, as clear from context. When “at least” is present before a series of numbers or a range, it is understood that “at least” can modify each of the numbers in the series or range.

As used herein, “no more than” or “less than” is understood as the value adjacent to the phrase and logical lower values or integers, as logical from context, to zero (if negative values are not possible). When “no more than” is present before a series of numbers or a range, it is understood that “no more than” can modify each of the numbers in the series or range.

As used herein, “up to” as in “up to 10” is understood as up to and including 10, i.e., 0, 1, 2, 3, 4, 5, 6, 7, 8, 9, or 10, in the context of non-negative integers.

Where a range of values is provided, it is understood that each intervening value (e.g., to the tenth of the unit of the lower limit unless the context clearly dictates otherwise) between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

### EXAMPLES

### Example 1: Detection of TLSs in Cohorts of Cancer Patients

The machine learning model for predicting the TLS status was cross-validated using a pan-cancer cohort of 289 H& E-stained Whole Slide Images (WSIs) obtained from 289 patients, one slide from each patient, from Institut Bergonid. On these WSIs, the TLS status was manually examined by expert pathologists and was annotated. The cohort was comprised of WSIs from 113 non-small cell lung cancer (NSCLC) patients (39.1%), 45 sarcoma patients (15.6%), 30 bladder cancer patients (10.4%), 26 colorectal cancer patients (9.0%), 10 kidney patients (3.7%), 10 head and neck cancer patients (3.7%), 9 ovarian cancer patients (3.1%), 7 liver cancer patients, 5 breast cancer patients, 5 gastro-intestinal stromal tumor cancer patients, 4 cervix cancer patients, 4 endometrial cancer patients, 4 stomach cancer patients, 3 thyroid cancer patients, 2 cholangiocarcinoma patients, 2 prostate cancer patients, 2 anal cancer patients, 2 vulvar cancer patients, 1 skin cancer patient, 1 parotid cancer patient, 1 digestive cancer patient, 1 penile cancer patient, 1 carcinoma of unknown primary cancer patient, and 1 esophageal cancer patient. A Deep Learning (DL) model was trained on the WSI to predict TLS status at the patient level (presence or absence of TLS).

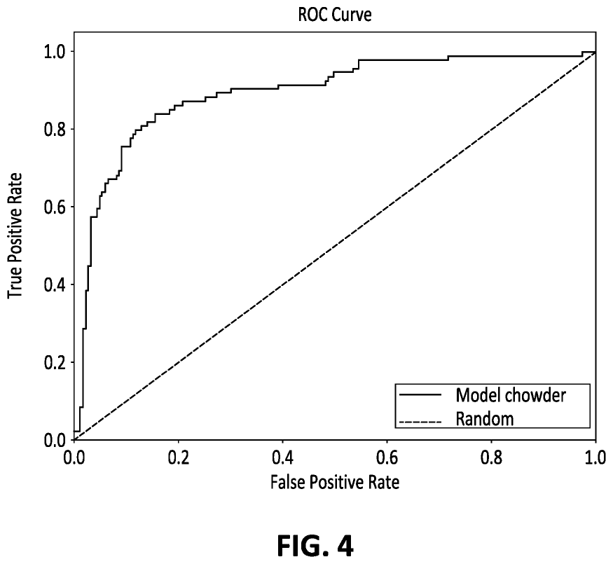

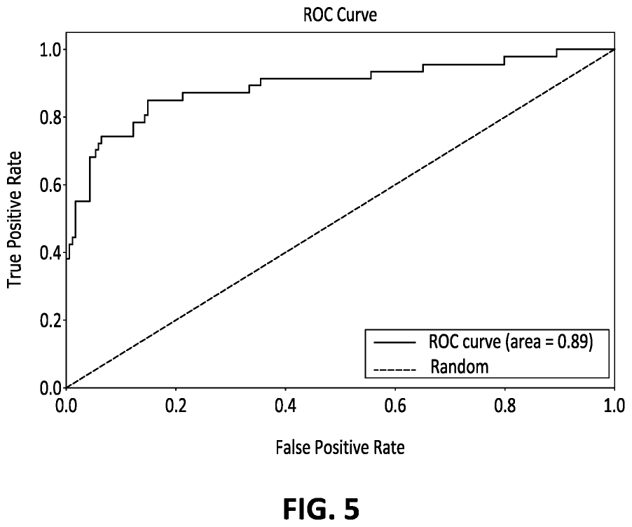

Models were evaluated using five-fold cross validation. The best performing DL model provided two main components—a predicted score (tile score) of TLS presence in small areas of the WSIs having a size of 112 μm×112 μm (i.e., one tile), followed by an aggregation at the patient level—with an ROC AUC score of 0.917 (standard deviation 0.036) (FIG. 4). The trained DL model provided the subject's TLS status with a sensitivity of 90% with a specificity of 68%; a sensitivity of 85% with a specificity of 85%; and a sensitivity of 80% with a specificity of 87%.

The transferability of the DL model was assessed using a validation cohort (PEMBROSARC) of 236 sarcoma WSIs (subjects), which included 47 WSIs (subjects) (19.9% of the entire cohort) with a positive TLS status (i.e., TLSs are present). The PEMBROSARC study is the first clinical trial implementing TLS status as an inclusion criteria (Italiano A. et al. 2022 Nat. Med. 28:1199-1206). The DL model detected the subject's TLS status with a ROC AUC score of 0.89, providing a sensitivity of 90% with specificity of 64%; a sensitivity of 85% with specificity of 88%; and a sensitivity of 80% with specificity of 86%. In sum, the study demonstrated the predictive power of DL models to detect the subject's TLS status based on images of H&E-stained histology slides. The DL models provided herein can be implemented in pathology labs and health care facilities as an efficient pre-screening tool for TLS status of subjects.

The foregoing discussion merely describes some exemplary embodiments of the present invention. One skilled in the art will readily recognize from such discussion, the accompanying drawings and the claims that various modifications can be made without departing from the spirit and scope of the invention.

1. A computer-implemented method for detecting a presence or absence of a tertiary lymphoid structure (TLS) in a subject, comprising:
receiving a digitalized histology image of a sample obtained from the subject;
tiling the histology image into a set of tiles;
extracting one or more feature vectors from each of said tiles, wherein each feature of said one or more feature vectors represents local descriptors of the tile; and
classifying the histology image for a TLS status using at least the plurality of feature vectors and a classification model that is trained with a training set of histology images having known TLS annotations, wherein the TLS status indicates the presence or absence of a TLS in the subject.

2. The computer-implemented method of claim 1, wherein the classifying comprises:
applying a first neural network to said one or more of the plurality of feature vectors, wherein said first neural network assigns a tile score to each tile of said set of tiles based on said one or more of the plurality of feature vectors, wherein the tile score represents a likelihood that said tile comprises a TLS; and
applying a second neural network to said each tile, wherein said second neural network aggregates subsets of said tile score of said set of tiles and determines the TLS status in the histology image, wherein:
said first neural network is trained using a training set of histology images comprising known local annotations of a presence or absence of a TLS at tile level; and
said first neural network and said second neural network are trained using a training set of histology images comprising known global annotations of a presence or absence of a TLS at histology image level.

3. (canceled)

4. (canceled)

5. The computer-implemented method of claim 1, further comprising detecting one or more locations in which a TLS is present in said histology image.

6. The computer-implemented method of claim 1, wherein each tile comprises a plurality of pixels, and wherein the method further comprises:
receiving a digitalized histology image of a sample obtained from the subject;
tiling the histology image into a set of tiles;
extracting segmentation mask from each tile;
applying a third neural network to each tile, wherein the third neural network detects pixel scores using extracted segmentation mask; and
detecting pixel-based TLS segmentation within the tile or the image, wherein said third neural network is trained using a training set of tiles comprising known pixel-based TLS segmentation mask within a tile.

7. The computer-implemented method of claim 4, wherein said third neural network assigns a pixel score to each pixel of each tile of said set of tiles and determines said pixel-based segmentation of a TLS within a tile, wherein the pixel score represents a likelihood that said pixel comprises a TLS.

8. (canceled)

9. The method of claim 1, wherein said extracting step is performed by ResNet50 neural network and/or Momentum Contrast (MoCo) or Momentum Contrast v2 (MoCo v2) algorithm.

10. (canceled)

11. (canceled)

12. The computer-implemented method of claim 1, wherein the training set of tiles and/or training set of histology images are digitalized images of histology sections of cancers of heterologous origins.

13. (canceled)

14. (canceled)

15. (canceled)

16. The computer-implemented method of claim 2, wherein the classifying step further comprises:
sorting said set of tiles by
picking tiles comprising highest TLS tile scores, and
picking tiles comprising lowest TLS tile scores.

17. The computer-implemented method of claim 1, comprising:
repeating all the steps of claim 1 in a plurality of histology images, and
processing the TLS status of the plurality of histology images, thereby detecting the presence or absence of a TLS in a subject.

18. The computer-implemented method of claim 1, wherein the histology image lacks local annotations of histopathological features.

19. (canceled)

20. A non-transitory machine readable medium having executable instructions to cause one or more processing units to perform a method of detecting a presence or absence of tertiary lymphoid structure (TLS) in a subject, comprising:
receiving a digitalized histology image of a sample obtained from the subject;
tiling the histology image into a set of tiles;
extracting a plurality of feature vectors from each of said tiles, wherein each feature of said one or more feature vectors represents local descriptors of the tile; and
classifying the histology image for a TLS status using at least the plurality of feature vectors and a classification model that is trained with a imaging training set having known TLS annotations, wherein the TLS status indicates the presence or absence of a TLS in the subject.

21. The non-transitory machine readable medium of claim 20, wherein the classifying comprises:
applying a first neural network to said one or more of the plurality of feature vectors, wherein said first neural network assigns a tile score to each tile of said set of tiles based on said one or more of the plurality of feature vectors, wherein the tile score represents a likelihood that said tile comprises a TLS; and
applying a second neural network to said each tile, wherein said second neural network aggregates subsets of said tile score of said set of tiles and determines the TLS status in the histology image, wherein:
said first neural network is trained using a training set of histology images comprising known local annotations of a presence or absence of a TLS at tile level; and
said first neural network and said second neural network are trained using a training set of histology images comprising known global annotations of a presence or absence of a TLS at histology image level.

22. (canceled)

23. (canceled)

24. The non-transitory machine readable medium of claim 20, further comprising
detecting one or more locations in which a TLS is present in said histology image.

25. The non-transitory machine readable medium of claim 20, wherein each tile comprises a plurality of pixels, and wherein the method further comprises:
receiving a digitalized histology image of a sample obtained from the subject;
tiling the histology image into a set of tiles;
extracting segmentation mask from each tile;
applying a third neural network to each tile, wherein the third neural network detects pixel scores using extracted segmentation mask; and
detecting pixel-based TLS segmentation within the tile or the image, wherein said third neural network is trained using a training set of tiles comprising known pixel-based TLS segmentation mask within a tile.

26. The non-transitory machine readable medium of claim 25, wherein said third neural network assigns a pixel score to each pixel of each tile of said set of tiles and determines said pixel-based segmentation of a TLS within a tile, wherein the pixel score represents a likelihood that said pixel comprises a TLS.

27. (canceled)

28. The non-transitory machine readable medium of claim 20, wherein said extracting step is performed by ResNet50 neural network and/or Momentum Contrast (MoCo) or Momentum Contrast v2 (MoCo v2) algorithm.

29. (canceled)

30. (canceled)

31. The non-transitory machine readable medium of claim 20, wherein the training set of tiles and/or training set of histology images are digitalized images of histology sections of cancers of heterologous origins.

32. (canceled)

33. (canceled)

34. (canceled)

35. The non-transitory machine readable medium of claim 20, wherein the classifying step further comprises:
sorting said set of tiles by
picking tiles comprising highest TLS tile scores, and
picking tiles comprising lowest TLS tile scores.

36. The non-transitory machine readable medium of claim 20, comprising:
repeating all the steps of claim 17 in a plurality of histology images, and
processing the TLS status of the plurality of histology images, thereby detecting the presence or absence of a TLS in a subject.

37. The non-transitory machine readable medium of claim 20, wherein the histology image lacks local annotations of histopathological features.

38. (canceled)
